(19) United States
(12) Patent Application Publication
(10) Pub. No.: US 2021/0177908 A1
(43) Pub. Date: Jun. 17, 2021

(54) ANABOLIC TARGETING STEM CELL GENE THERAPY FOR OSTEOPOROSIS

(71) Applicants: LOMA LINDA UNIVERSITY, Loma Linda, CA (US); FACULTY PHYSICIANS AND SURGEONS OF LOMA LINDA UNIVERSITY SCHOOL OF MEDICINE, Loma Linda, CA (US); THE UNITE STATES OF AMERICA AS REPRESENTED BY UNTED STATES DEPARTMENT OF VETERANS AFFAIRS, Washington, DC (US)

(72) Inventors: Zhang XIAOBING, Loma Linda, CA (US); David BAYLINK, Loma Linda, CA (US); Wanqui CHEN, Loma Linda, CA (US); Kin-Hing William LAU, c, DC (US)

(21) Appl. No.: 17/256,509

(22) PCT Filed: Jun. 28, 2019
(86) PCT No.: PCT/US19/39969

§ 371 Date: Dec. 28, 2020

Related U.S. Application Data
(60) Provisional application No. 62/691,541, filed on Jun. 28, 2018.

(51) Int. Cl. A61K 35/28; A61P 19/10; C07K 14/49; C07K 14/50; C07K 5/09

(52) U.S. Cl. CPC A61K 35/28; A61P 19/10; A61K 38/00; C07K 14/50; C07K 5/0817; C07K 14/49

(57) ABSTRACT

In one aspect, anabolic agent fusion proteins and compositions comprising anabolic agent fusion proteins are provided. In some embodiments, the anabolic agent fusion protein comprises a platelet derived growth factor (PDGF) or a fibroblast growth factor (FGF) and an Asp-Ser-Ser tripeptide (DSS) repeat sequence. In another aspect, methods of promoting bone growth and methods of treating a fracture using anabolic agent fusion proteins and compositions comprising anabolic agent fusion proteins are provided.

### BACKGROUND OF THE INVENTION

Osteoporosis is a common disease leading to 10 million fractures worldwide annually. Restorative anabolic therapy is regarded as a promising treatment for the treatment of osteoporosis, as well as other types of pathologic bone loss or injury. However, the two candidate anabolic drugs that are known in the field have significant drawbacks: Parathyroid hormone (1-34) (PTH(1-34)) is incapable of totally regenerating the skeleton, while the anti-sclerostin antibody romozumab has an anabolic effect that dissipates after one year and can cause cardiovascular off-target effects. Accordingly, there remains a need for effective anabolic agents for promoting bone formation.

### BRIEF SUMMARY OF THE INVENTION

In one aspect, engineered cells that expresses an anabolic agent fusion protein are provided. In some embodiments, the anabolic agent fusion protein comprises a platelet derived growth factor (PDGF) or a fibroblast growth factor (FGF) and an Asp-Ser-Ser tripeptide (DSS) repeat sequence.

In some embodiments, the anabolic agent fusion protein comprises PDGF. In some embodiments, the PDGF is human PDGF. In some embodiments, the PDGF is a homodimer of PDGF subunit B (PDGF-BB).

In some embodiments, the anabolic agent fusion protein comprises FGF. In some embodiments, the FGF is modified FGF.

In some embodiments, the DSS repeat sequence has from 2 repeats to 8 repeats. In some embodiments, the DSS repeat sequence has 6 repeats (DSS6).

In some embodiments, the anabolic agent fusion protein comprises PDGF fused to DSS6.

In some embodiments, the engineered cell comprises an expression cassette comprising a promoter operably linked to a polynucleotide that encodes the anabolic agent fusion protein. In some embodiments, the promoter is a promoter for a housekeeping gene. In some embodiments, the promoter is a full-length or truncated form of a PGK promoter.

In some embodiments, a polynucleotide encoding the anabolic agent fusion protein is integrated into the genome of the engineered cell.

In some embodiments, the cell is a stem cell. In some embodiments, the cell is a hematopoietic stem cell. In some embodiments, the cell is a mesenchymal stem cell.

In another aspect, pharmaceutical compositions comprising an engineered cell as disclosed herein are provided. In some embodiments, the pharmaceutical composition comprises the engineered cell and a pharmaceutically acceptable carrier. In some embodiments, the pharmaceutical composition further comprises serum albumin.

In another aspect, pharmaceutical compositions comprising an anabolic agent fusion protein and a pharmaceutically acceptable carrier are provided. In some embodiments, the anabolic agent fusion protein comprises a PDGF or an FGF fused to a DSS repeat sequence. In some embodiments, the anabolic agent fusion protein comprises PDGF. In some embodiments, the PDGF is human PDGF. In some embodiments, the PDGF is PDGF-BB. In some embodiments, the anabolic agent fusion protein comprises FGF. In some embodiments, the FGF is modified FGF. In some embodiments, the DSS repeat sequence has from 2 repeats to 8 repeats. In some embodiments, the DSS repeat sequence is DSS6. In some embodiments, the anabolic agent fusion protein comprises PDGF fused to DSS6.

In yet another aspect, methods of treating a fracture are provided. In some embodiments, the method comprises administering to a subject having a fracture an anabolic agent fusion protein, wherein the anabolic agent fusion protein comprises PDGF or FGF fused to a DSS repeat sequence. In some embodiments, the anabolic agent fusion protein comprises PDGF. In some embodiments, the PDGF is human PDGF. In some embodiments, the PDGF is PDGF-BB. In some embodiments, the anabolic agent fusion protein comprises FGF. In some embodiments, the FGF is modified FGF. In some embodiments, the DSS repeat sequence has from 2 repeats to 8 repeats. In some embodiments, the DSS repeat sequence is DSS6. In some embodiments, the anabolic agent fusion protein comprises PDGF fused to DSS6.

In some embodiments, the method comprises administering a recombinant or synthetic form of the anabolic agent fusion protein. In some embodiments, the method comprises locally administering the anabolic agent fusion protein to the site of a fracture. In some embodiments, the anabolic agent fusion protein is administered by injection. In some embodiments, the anabolic agent fusion protein is administered by micropump. In some embodiments, the fracture is a delayed union fracture, an established nonunion fracture, or a simple fracture. In some embodiments, the fracture is a fracture of a tibia, fibula, femur, radius, ulna, tarsal, metatarsal, carpal, metacarpal, vertebra, clavicle, or pelvis.

In some embodiments, administering the anabolic agent fusion protein accelerates delayed fracture healing. In some embodiments, a single dose of the anabolic agent fusion protein is administered to the subject.

In another aspect, methods of increasing bone growth in a subject are provided. In some embodiments, the method comprises administering to the subject a therapeutically effective amount of engineered cells that express an anabolic agent fusion protein or a pharmaceutical composition comprising the engineered cells, wherein the anabolic agent fusion protein comprises PDGF or FGF fused to a DSS repeat sequence.

In some embodiments, the anabolic agent fusion protein comprises PDGF. In some embodiments, the PDGF is human PDGF. In some embodiments, the PDGF is PDGF-BB. In some embodiments, the anabolic agent fusion protein comprises FGF. In some embodiments, the FGF is modified FGF. In some embodiments, the DSS repeat sequence has from 2 repeats to 8 repeats. In some embodiments, the DSS repeat sequence is DSS6. In some embodiments, the anabolic agent fusion protein comprises PDGF fused to DSS6.

In some embodiments, the engineered cell is a stem cell. In some embodiments, the stem cell is a hematopoietic stem cell or a mesenchymal stem cell. In some embodiments, the engineered cell is autologous to the subject.

In some embodiments, the engineered cell is administered locally. In some embodiments, the engineered cell is administered by injection. In some embodiments, the engineered cell is administered systemically. In some embodiments, the engineered cell is administered intravenously.

In some embodiments, the subject has osteoporosis. In some embodiments, the subject has severe osteoporosis.

In yet another aspect, methods of increasing bone growth in a subject are provided. In some embodiments, the method comprises locally administering to the subject an anabolic agent fusion protein that comprises PDGF or FGF fused to a DSS repeat sequence, wherein the anabolic agent fusion protein is locally administered to a site of bone loss.

In some embodiments, the anabolic agent fusion protein comprises PDGF. In some embodiments, the PDGF is human PDGF. In some embodiments, the PDGF is PDGF-BB. In some embodiments, the anabolic agent fusion protein comprises FGF. In some embodiments, the FGF is modified FGF. In some embodiments, the DSS repeat sequence has from 2 repeats to 8 repeats. In some embodiments, the DSS repeat sequence is DSS6. In some embodiments, the anabolic agent fusion protein comprises PDGF fused to DSS6.

In some embodiments, the method comprises administering a recombinant or synthetic form of the anabolic agent fusion protein. In some embodiments, the anabolic agent fusion protein is administered by injection.

In some embodiments, the subject has osteoporosis. In some embodiments, the subject has severe osteoporosis.

### BRIEF DESCRIPTION OF THE DRAWINGS

FIG. 1A-1B. (A) DSS6 endows a bone surface binding ability to GFP. Bone chips were incubated for 1 hour with 50 ng/ml of GFP or GFP-DSS6. Images were taken after 2 washes with PBS. (B) Administration of PDGFB-DSS6 promotes bone formation. Mice were injected with PDGF-B or PDGFB-DSS6 twice a week for three weeks, then two-color dynamic histomorphometry of the midshaft femoral endosteum was performed to examine mineral apposition rate, using Xylenol (red) and Calcein (green) in PBS.

FIG. 2A-2C. (A-B) microCT analysis of femur at 10 weeks after transplantation of Sca1-GFP, Sca1-PDGF or Sca1-PDGF-DSS6 into OVX mice. (C) microCT analysis of tail vertebra of recipient mice receiving either PDGFB-transduced Sca-1⁺ cells (PDGFB) or GFP-transduced Sca1⁺ cells (Control-GFP) after 12 weeks post-transplantation.

FIG. 3. Cortical porosity at 10 weeks after transplantation of Sca1-GFP, Sca1-PDGFB or Sca1-PDGF-DSS6 into OVX mice.

FIG. 4. PDGFB-DSS6 protein promotes bone formation in lumbar vertebrae in OVX mice. At 1 month after ovariectomy, mice were received with PBS or PDGFB-DSS6 (0.5 mg/kg or 5 mg/kg) i.v. thrice per week for 4 weeks. Representative von Kossa staining images from L3 vertebrae showed increased trabecular bone formation following PDGFB-DSS6 treatment.

FIG. 5. Schematics of experimental design. After OVX or sham surgery animals undergo irradiation and transplantation with Sca1⁺ cells that were transduced with lenti GFP PGK PDFGB or GFP PGK PDGFB-DSS6, and bone tissues were analyzed 10 weeks later.

FIG. 6. Bone marrow transplantation and % GFP engraftment. As mentioned in the methods section, osteoporosis was induced in mice by OVX surgery. Two weeks after surgery, the C57/BL6 mice were divided into 3 groups, each with 6-7 mice, and transplanted with Sca1⁺ cells that were transduced with 1) Lenti GFP control, 2) Lenti GFP PDGF (wild-type), and 3) Lenti GFP PDGF-DSS6. To ensure engraftment of hematopoietic stem/progenitor cells, mice were myeloablated by irradiation at 8 Gy before transplantation. Ten weeks after transplantation the % engraftment was evaluated by analyzing the bone marrow cells for GFP fluorescence. Data are means±SEM. **P<0.01, ns−not significant.

FIG. 7. High Serum ALP level was observed in the PGK-PDGFB DSS6 treated animals, but not in the PGK-PDGF treated animals. Data are means±SEM. **P<0.01, ***P<0.001.

FIG. 8. Representative X-ray pictures of femurs harvested from mice received GFP control, PDGF or PDGF-DSS6 overexpressing Sca1⁺ cells. In each group, 6-7 mice were conducted OVX to induce osteoporosis, followed by hematopoietic stem/progenitor cell transplantation 2 weeks later. Animals were analyzed at 10 weeks after transplantation.

FIGS. 9A-9E. MicroCT three dimensional trabecular bone structure analysis of femurs from OVX osteoporosis mouse after treatment with PDGF or PDGF-DSS6. PDGF-DSS6 is a fusion protein of PDGFB and bone-surface binding peptide DSS6. Specimen were analyzed by microCT at 10 weeks after transplantation (n=6-7). The following parameters of new bone formation from the microCT analysis are shown: bone volume/trabecular volume (BV/TV); connectivity density (Conn. Density); trabecular number (Tb.N); trabecular thickness (Tb.Th), and cortical porosity. Data are means±SEM. ****P<0.0001.

FIG. 10. The PGK-PDGFB-DSS6 treatment increases bone strength and reduces the cortical porosity. Representative loading force displacement graph presented. Three-point bending test was used to measure bone strength at the midshaft of the femur. Maximum load-to-failure on PGK-PDGFB-DSS6 treated femurs was significantly greater than that of the other groups. Data are means±SEM. **P<0.01, ***P<0.001, ****P<0.0001.

FIG. 11. Bone histological staining for Von-Kossa reveal that the PGK-PDGFB-DSS6 treatment increases bone regeneration. Increased von Kossa stained mineralized bone is seen in the diaphysis as well as the metaphysis.

### DETAILED DESCRIPTION OF THE INVENTION

### I. Introduction

Bone has the innate capacity to regenerate. Although therapy with anabolic agents has been proposed as a way to increase bone strength and regenerate the skeleton, proposed anabolic therapies to date have been ineffective or are limited in their potential applications. For example, the FDA-approved therapy PTH1-34 does not have a sufficiently sustained robust action to regenerate the skeleton to normalize bone strength. See, Hegde et al., Osteoporosis International, 2016, 27:861-871. Stem cell-based gene therapy has also been proposed for increasing bone formation. It was previously reported that hematopoietic stem cells engineered to overexpress the mitogenic protein platelet derived growth factor (PDGF), when transplanted into mice, resulted in increased lamellar bone matrix formation at the endosteum. However, the transplanted mice also exhibited secondary hyperparathyroidism and severe osteomalacia. Chen et al., PNAS, 2015, 112:E3893-E3900.

In one aspect, the present disclosure provides anabolic agent fusion proteins comprising an anabolic agent fused to a DSS calcium-binding peptide and cells (e.g., hematopoietic stem cells) engineered to express anabolic agent fusion proteins. The anabolic agent fusion proteins and engineered cells disclosed herein have the advantage of being able to specifically target the bone surface, due to the presence of the DSS peptide, thereby allowing engineered hematopoietic stem cells expressing the fusion protein to localize expression of the anabolic agent to the marrow space with minimal diffusion into the circulation. The specific targeting to bone surface that is achieved using the anabolic agent fusion protein also minimizes off-target effects, such as fibrosis in soft tissues that has been reported with systemic administration of the anabolic agent PDGF. The anabolic agent fusion proteins disclosed herein also are extraordinarily effective in promoting bone growth. As shown in the Examples section below, administration of engineered hematopoietic stem cells expressing a PDGF-DSS6 fusion protein resulted in a more than 10-fold increase in bone formation, as compared to PDGF-expressing hematopoietic stem cells, in a mouse model of osteoporosis.

### II. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

As used herein, “PDGF” refers to platelet derived growth factor, a growth factor that regulates cell growth and division. There are multiple genes that encode for a PDGF polypeptide; in humans, there are two PDGF polypeptides, PDGF subunit A (PDGF-A) and PDGF subunit B (PDGF-B). Exemplary mRNA and protein sequences for PDGF-A are set forth as GenBank Accession Nos. NM_002607.5 and NP_002598.4, respectively, each of which are incorporated herein by reference. Exemplary mRNA and protein sequences for PDGF-B are set forth as GenBank Accession Nos. NM_011057.3 and NP_035187.2, respectively, and as GenBank Accession Nos. NM_002608.2, and NP_002599, respectively, each of which are incorporated herein by reference. Other mammalian PDGFB proteins include, but are not limited to mouse PDGF-B (GenBank Accession Nos. XM_006520591.1 (mRNA), XP_00652065301 (protein)), cow PDGF-B (GenBank Accession Nos. NM_001017953.2 (mRNA), NP_001017953.2 (protein)), monkey PDGF-B (GenBank Accession Nos. XM_001097395.2 (mRNA), XP_001097395 (protein)), dog PDGF-B (GENBANK Accession No. NM_001003383.1 (mRNA), NP_001003383.1 (protein), and rat PDGF-B (GENBANK Accession No. L40991.1 (mRNA), AAA70048.1 (protein)). Biologically active PDGF protein exists as a disulfide-linked dimer of two polypeptide chains, and can be a homodimer of two of the same PDGF polypeptide chain subunits (e.g., two subunits of PDGF-A or two subunits of PDGF-B) or a heterodimer of two different PDGF polypeptide chain isoforms (e.g., one subunit of PDGF-A and one subunit of PDGF-B). In some embodiments, a PDGF protein comprises a polypeptide that has at least 70%, at least 75% at least 80%, at least 85%, at least 90%, at least 91%, at least 92%, at least 93%, at least 94%, at least 95%, at least 96%, at least 97%, at least 98%, or at least 99% identity to the PDGF-A polypeptide of NCBI GenBank Accession No. NP_002598.4. In some embodiments, a PDGF protein comprises a polypeptide that has at least 70%, at least 75% at least 80%, at least 85%, at least 90%, at least 91%, at least 92%, at least 93%, at least 94%, at least 95%, at least 96%, at least 97%, at least 98%, or at least 99% identity to the PDGF-B polypeptide of NCBI GenBank Accession No. NP_035187.2 or NP_002599.

As used herein, “FGF” refers to fibroblast growth factor, a growth factor that is involved in multiple cellular processes including cell growth, division, and survival. The FGF protein family is a large family for which more than 20 members are currently known; the FGF family is subdivided into more closely related sub-families. FGF proteins include acidic FGF (aFGF, also known as FGF-1), basic FGF (b-FGF, also known as modified FGF-2), FGF-2, FGF-3, FGF-4 FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23. As used herein, “FGF” includes unmodified and modified forms of an FGF, e.g., basic or unmodified FGF-2 and modified FGF-2. Some FGF proteins are believed to participate in bone regeneration, such as FGF-2, FGF-9, and FGF-18. See, Charoenlarp et al., Inflamm Regen, 2017, 37:10. In some embodiments, the FGF protein is modified FGF-2. Exemplary mRNA and protein sequences for human FGF-2 are set forth as GenBank Accession Nos. NM_002006.5 and NP_001997.5, respectively, each of which are incorporated herein by reference. In some embodiments, an FGF protein comprises a polypeptide that has at least 70%, at least 75% at least 80%, at least 85%, at least 90%, at least 91%, at least 92%, at least 93%, at least 94%, at least 95%, at least 96%, at least 97%, at least 98%, or at least 99% identity to the FGF-2 polypeptide of NCBI GenBank Accession No. NP_001997.5.

The terms “identical” or “percent identity,” in the context of two or more polynucleotide or polypeptide sequences, refer to two or more sequences that are the same or have a specified percentage of amino acid residues or nucleotides that are the same (e.g., about 60% identity, preferably 65%, 70%, 75%, 80%, 85%, 90%, 91%, 92%, 93%, 94%, 95%, 96%, 97%, 98%, 99%, or higher identity) over a specified region. Methods for comparing polynucleotide or polypeptide sequences and determining percent identity are described in the art. See, e.g., Roberts et al., BMC Bioinformatics, 7:382, 2006, incorporated by reference herein.

The terms “nucleic acid” and “polynucleotide” are used interchangeably herein and refer to deoxyribonucleotides or ribonucleotides and polymers thereof in either single- or double-stranded form, and complements thereof. In some embodiments, the polynucleotide is DNA (e.g., genomic DNA or cDNA). In some embodiments, the polynucleotide is RNA (e.g., mRNA). Unless otherwise indicated, a particular nucleic acid sequence also implicitly encompasses conservatively modified variants thereof (e.g., degenerate codon substitutions), polymorphic variants (e.g., SNPs), splice variants, and nucleic acid sequences encoding truncated forms of proteins, complementary sequences, as well as the sequence explicitly indicated.

The terms “protein” and “polypeptide” are used interchangeably herein and refer to a polymer of amino acid residues. As used herein, the terms encompass amino acid chains of any length, including full-length proteins and truncated proteins.

The term “promoter,” as used herein, refers to a polynucleotide sequence capable of driving transcription of a coding sequence in a cell. In some embodiments, a promoter includes cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (e.g., turn on/off, regulate, modulate) gene transcription. A “constitutive promoter” is one that is capable of initiating transcription in nearly all tissue types, whereas a “tissue-specific promoter” initiates transcription only in one or a few particular tissue types.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., the promoter is from a different gene in the same species).

As used herein, a “subject” is a mammal, in some embodiments, a human. Mammals can also include, but are not limited to, farm animals (e.g., cows, pigs, horses, chickens, etc.), sport animals, pets, primates, horses, dogs, cats, mice and rats.

As used herein, the terms “treatment,” “treating,” and “treat” refer to any indicia of success in the treatment or amelioration of an injury, disease, or condition, including any objective or subjective parameter such as abatement; remission; diminishing of symptoms or making the injury, disease, or condition more tolerable to the subject; slowing in the rate of degeneration or decline; making the final point of degeneration less debilitating; and/or improving a subject's physical or mental well-being.

As used herein, a “therapeutic amount” or a “therapeutically effective amount” of an agent (e.g., an anabolic agent fusion protein, an engineered cell that expresses an anabolic agent fusion protein, or a pharmaceutical composition comprising an anabolic agent fusion protein or engineered cell as described herein) is an amount of the agent that prevents, alleviates, abates, or reduces the severity of symptoms of a disease (e.g., osteoporosis) in a subject. For example, for the given parameter, a therapeutically effective amount will show an increase or decrease of therapeutic effect of at least 5%, 10%, 15%, 20%, 25%, 40%, 50%, 60%, 75%, 80%, 90%, or at least 100%. Therapeutic efficacy can also be expressed as “fold” increase or decrease. For example, a therapeutically effective amount can have at least a 1.2-fold, 1.5-fold, 2-fold, 5-fold, or more effect over a control.

The terms “administer,” “administered,” or “administering,” as used herein, refer to introducing an agent (e.g., an anabolic agent fusion protein, an engineered cell that expresses an anabolic agent fusion protein, or a pharmaceutical composition comprising an anabolic agent fusion protein or engineered cell as described herein) into a subject or patient, such as a human. As used herein, the terms encompass both direct administration, (e.g., self-administration or administration to a patient by a medical professional) and indirect administration (e.g., the act of prescribing a compound or composition to a subject).

As used herein, the term “pharmaceutical composition” refers to a composition suitable for administration to a subject. In general, a pharmaceutical composition is sterile, and preferably free of contaminants that are capable of eliciting an undesirable response with the subject. Pharmaceutical compositions can be designed for administration to subjects in need thereof via a number of different routes of administration, including oral, intravenous, buccal, rectal, parenteral, intraperitoneal, intradermal, intratracheal, intramuscular, subcutaneous, inhalational, transdermal, and the like.

### III. Anabolic Targeting Constructs and Fusion Proteins

In one aspect, the present disclosure provides anabolic agent fusion proteins, isolated nucleic acids comprising a polynucleotide encoding an anabolic agent fusion protein, and constructs (e.g., expression cassettes) comprising a polynucleotide encoding an anabolic agent fusion protein. In some embodiments, the anabolic agent fusion protein comprises a mitogenic protein and an Asp-Ser-Ser tripeptide (DSS) repeat sequence. A “mitogenic protein” as used herein is a protein that promotes cell division. Mitogenic proteins are known in the art, and include, for example, platelet derived growth factor (PDGF), fibroblast growth factor (FGF), and bone morphogenetic protein (BMP). In some embodiments, the anabolic agent fusion protein comprises a PDGF or an FGF fused to a DSS repeat sequence.

In one aspect, fusion proteins comprising an anabolic agent fused to a DSS repeat sequence are provided. In some embodiments, the anabolic agent is a mitogenic protein. In some embodiments, the anabolic agent is a PDGF or an FGF. In some embodiments, the fusion protein comprises PDGF. In some embodiments, the PDGF is human PDGF. In some embodiments, the PDGF is a homodimer of PDGF subunit A (PDGF-AA). In some embodiments, the PDGF is a homodimer of PDGF subunit B (PDGF-BB). In some embodiments, the PDGF is a heterodimer of PDGF subunits A and B (PDGF-AB).

### Anabolic Agents

In some embodiments, the anabolic agent is a mitogenic protein, such as a PDGF, an FGF, or a BMP. In some embodiments, the anabolic agent is a human mitogenic protein.

In some embodiments, the PDGF (e.g., human PDGF, e.g., human PDGF-B) comprises a full-length PDGF protein, a peptide fragment having the functional activity of a full-length PDGF protein, or a peptide mimetic having the functional activity of a full-length PDGF protein. In some embodiments, the PDGF (e.g., human PDGF, e.g., human PDGF-B) comprises a full-length PDGF protein. In some embodiments, the PDGF comprises a sequence that is identical to a naturally-occurring (i.e., wild-type) PDGF. In some embodiments, the PDGF comprises one or more mutations (e.g., insertions, deletions, or substitutions) relative to a wild-type PDGF polypeptide. In some embodiments, a PDGF protein comprises a polypeptide that has at least 70%, at least 75% at least 80%, at least 85%, at least 90%, at least 91%, at least 92%, at least 93%, at least 94%, at least 95%, at least 96%, at least 97%, at least 98%, or at least 99% identity to the PDGF-B polypeptide of NCBI GenBank Accession No. NP_035187.2 or NP_002599. In some embodiments, the PDGF (e.g., human PDGF, e.g., human PDGF-B) comprises a peptide fragment or peptide mimetic having the functional activity of a full-length PDGF protein. PDGF peptide fragments and mimetics are known in the art. See, e.g., Duan et al., J. Biol. Chem, 1991, 266:413-418; Lin et al., Growth Factors, 2007, 25:87-93.

In some embodiments, the fusion protein comprises FGF (e.g., a basic or unmodified FGF or a modified FGF). In some embodiments, the FGF is human FGF. In some embodiments, the FGF is FGF-1, FGF-2, FGF-3, FGF-4 FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, or FGF-23. In some embodiments, the FGF is FGF-2, e.g., human FGF-2. In some embodiments, the FGF is a modified FGF, e.g., modified FGF-2. Modified FGFs such as modified forms of FGF-2 are disclosed in the art. See, e.g., Hall et al., Molecular Therapy, 2007, 15-1881-1889.

In some embodiments, the FGF (e.g., human FGF, e.g., human FGF-2) comprises a full-length FGF protein, a peptide fragment having the functional activity of a full-length FGF protein, or a peptide mimetic having the functional activity of a full-length FGF protein. In some embodiments, the FGF (e.g., human FGF, e.g., human FGF-2) comprises a full-length FGF protein. In some embodiments, the FGF (e.g., human FGF, e.g., human FGF-2) comprises a sequence that is identical to a naturally-occurring (i.e., wild-type) FGF. In some embodiments, the FGF comprises one or more mutations (e.g., insertions, deletions, or substitutions) relative to a wild-type FGF polypeptide. In some embodiments, an FGF protein comprises a polypeptide that has at least 70%, at least 75% at least 80%, at least 85%, at least 90%, at least 91%, at least 92%, at least 93%, at least 94%, at least 95%, at least 96%, at least 97%, at least 98%, or at least 99% identity to the FGF-2 polypeptide of NCBI GenBank Accession No. NP_001997.5. In some embodiments, the FGF is a modified FGF. In some embodiments, the FGF comprises a peptide fragment or peptide mimetic having the functional activity of a full-length FGF protein. FGF peptide fragments and mimetics are known in the art. See, e.g., Lin et al., Intl Mol Med, 2006, 17:833-839.

In some embodiments, the anabolic agent fusion protein (e.g., a PDGF or an unmodified or modified FGF fused to a DSS repeat sequence) is in a recombinant form. In some embodiments, the anabolic agent fusion protein (e.g., a PDGF or an unmodified or modified FGF fused to a DSS repeat sequence) is in a synthetic form.

### DSS Repeat Sequences

In some embodiments, the anabolic agent fusion protein comprises an Asp-Ser-Ser tripeptide (DSS) repeat sequence. The DSS peptide is a peptide that specifically binds to calcified surfaces. See, Yarbrough et al., Calcif Tissue Int, 2010, 86:58-66; Zhang et al., Nat Med, 2012, 18:307-314; Without being bound to a particular theory, it is believed that the presence of the DSS repeat sequence targets the delivery of the anabolic agent fusion protein to bone surfaces, such as sites of bone loss or bone injury in a subject.

In some embodiments, the DSS repeat sequence has from 2 repeats, e.g., at least 3 repeats, at least 4 repeats, at least 5 repeats, or at least 6 repeats. In some embodiments, the DSS repeat sequence has from 2 repeats to 8 repeats, or has from 4 repeats to 8 repeats, or has from 5 repeats to 7 repeats. In some embodiments, the DSS repeat sequence has 2 repeats (DSS2), 3 repeats (DSS3), 4 repeats (DSS4), 5 repeats (DSS5), 6 repeats (DSS6), 7 repeats (DSS7), or 8 repeats (DSS8).

In some embodiments, the anabolic agent fusion protein comprises PDGF (e.g., human PDGF) fused to a DSS repeat sequence having from 2 repeats to 8 repeats (e.g., DSS6). In some embodiments, the anabolic agent fusion protein comprises PDGF-B (e.g., human PDGF-B) fused to a DSS repeat sequence having from 2 repeats to 8 repeats (e.g., DSS6).

### Nucleic Acids, Expression Cassettes, and Vectors

In another aspect, isolated nucleic acids comprising a polynucleotide encoding an anabolic agent fusion protein are provided. In some embodiments, the polynucleotide encodes a fusion protein comprising a mitogenic protein (e.g., PDGF or unmodified or modified FGF) fused to a DSS repeat sequence. In some embodiments, the polynucleotide encodes a human PDGF fused to a DSS repeat sequence. In some embodiments, the PDGF is human PDGF-B. In some embodiments, the PDGF (e.g., human PDGF, e.g., human PDGF-B) comprises a sequence that is identical to a naturally-occurring (i.e., wild-type) PDGF. In some embodiments, the PDGF (e.g., human PDGF, e.g., human PDGF-B) comprises one or more mutations (e.g., insertions, deletions, or substitutions) relative to a wild-type PDGF polypeptide. In some embodiments, the PDGF has at least 70%, at least 75% at least 80%, at least 85%, at least 90%, at least 91%, at least 92%, at least 93%, at least 94%, at least 95%, at least 96%, at least 97%, at least 98%, or at least 99% identity to the PDGF-B polypeptide of NCBI GenBank Accession No. NP_035187.2 or NP_002599. In some embodiments, the polynucleotide encodes a human FGF fused to a DSS repeat sequence. In some embodiments, the FGF is human FGF-2. In some embodiments, the FGF (e.g., human FGF, e.g., human FGF-2) comprises a sequence that is identical to a naturally-occurring (i.e., wild-type) FGF. In some embodiments, the FGF (e.g., human FGF, e.g., human FGF-2) comprises one or more mutations (e.g., insertions, deletions, or substitutions) relative to a wild-type FGF polypeptide. In some embodiments, the FGF has at least 70%, at least 75% at least 80%, at least 85%, at least 90%, at least 91%, at least 92%, at least 93%, at least 94%, at least 95%, at least 96%, at least 97%, at least 98%, or at least 99% identity to the FGF-2 polypeptide of NCBI GenBank Accession No. NP_001997.5. In some embodiments, the polynucleotide encodes a modified FGF, e.g., a modified FGF-2.

In some embodiments, the polynucleotide encodes a fusion protein comprising a mitogenic protein (e.g., PDGF or unmodified or modified FGF) fused to a DSS repeat sequence having at least 2 repeats, e.g., at least 3 repeats, at least 4 repeats, at least 5 repeats, or at least 6 repeats. In some embodiments, the DSS repeat sequence has from 2 repeats to 8 repeats, or has from 4 repeats to 8 repeats, or has from 5 repeats to 7 repeats. In some embodiments, the DSS repeat sequence has 2 repeats (DSS2), 3 repeats (DSS3), 4 repeats (DSS4), 5 repeats (DSSS), 6 repeats (DSS6), 7 repeats (DSS7), or 8 repeats (DSS8). In some embodiments, the DSS repeat sequence is DSS6. In some embodiments, the polynucleotide encodes a fusion protein comprising PDGF (e.g., human PDGF) fused to a DSS repeat sequence having from 2 repeats to 8 repeats (e.g., DSS6). In some embodiments, the polynucleotide encodes a fusion protein comprising PDGF-B (e.g., human PDGF-B) fused to a DSS repeat sequence having from 2 repeats to 8 repeats (e.g., DSS6).

In some embodiments, the polynucleotide encoding the anabolic agent fusion protein is operably linked to one or more expression control elements. As used herein, an “expression control element” is a nucleic acid sequence that regulates the expression of a heterologous nucleic acid sequence to which it is operatively linked. Expression control elements include, for example, promoters, enhancers, repressor elements, transcription terminators, a start codon (ATG) in front of a protein-encoding gene, splicing signals for introns, and stop codons. In some embodiments, the polynucleotide encoding the anabolic agent fusion protein is operably linked to a heterologous promoter.

In some embodiments, the promoter is a constitutively active promoter. In some embodiments, the promoter is a strong promoter. In some embodiments, the promoter is an inducible promoter, e.g., a tetracycline-inducible promoter. In some embodiments, the promoter is a tissue-specific promoter, e.g., a stem cell-specific promoter, such as but not limited to a stem cell antigen 1 (SCA1) promoter, a hemoglobin promoter, or a CD34 promoter. In some embodiments, the promoter is a promoter from a housekeeping gene, e.g., a GAPDH promoter, actin promoter, or cyclophilin promoter. In some embodiments, the promoter is from a mid-level housekeeping gene. Examples of suitable promoters include, but are not limited to, phosphoglycerate Kinase-1 (PGK) promoter, P200 promoter, beta-globin promoter, human cytomegalovirus (CMV) promoter, Murine Stem Cell Virus (MSV) promoter, simian virus 40 (SV40) early promoter, mouse mammary tumor virus promoter, Moloney virus promoter, avian leukemia virus promoter, Epstein-Barr virus immediate early promoter, human elongation factor-1 alpha (EFlalpha) promoter, ubiquitous chromatin opening elements (UCOE) promoter, metallothionein promoter, retrovirus long terminal repeat; adenovirus late promoter, vaccinia virus 7.5K promoter, or spleen focus-forming virus (SFFV) promoter. In some embodiments, the promoter is a full-length or truncated form of a PGK promoter. In some embodiments, the promoter is a SCA1 promoter.

In some embodiments, the polynucleotide further comprises one or more elements for turning off expression of the anabolic agent fusion protein. In some embodiments, the element comprises a suicide gene, such as HSV thymidine kinase (HSV-TK). See, Painter et al., Cancer Sci, 2005, 96:607-613. In such embodiments, if an engineered cell (e.g., an engineered HSC) comprises the polynucleotide, the cell can be killed by administration of ganciclovir (GCV). HSV-TK converts GCV into a toxic product and therefore allows selective elimination of TK+cells. An exemplary working concentration of GCV is 10-100 mg/kg/day for 7-21 days. In some embodiments, expression of the anabolic agent fusion protein can be regulated using a TET-On system. In this embodiment, administration of doxycycline (Dox) can induce secretion of the fusion protein from transduced cells. Doxycycline is added, for example at a concentration of 1-1000 ng/ml. In some embodiments, expression of the anabolic agent fusion protein can be regulated using a tamoxifen-inducible promoter system. In this embodiment, administration of tamoxifen can induce secretion of the fusion protein from transduced cells. Tamoxifen is added, for example, at a concentration of 1-1,000 mg/ml. In some embodiments, expression of the anabolic agent fusion protein can be regulated using an ecdysone receptor-inducible promoter system. In this embodiment, administration of ecdysone can induce secretion of the fusion protein from transduced cells. In some embodiments, the polynucleotide encoding the anabolic agent fusion protein and the suicide gene (e.g., HSV-TK) are expressed using a bicistronic vector.

In some embodiments, expression cassettes are provided that comprise a polynucleotide encoding an anabolic agent fusion protein operably linked to one or more expression control elements, such as a promoter. In some embodiments, vectors comprising an expression cassette that comprise a polynucleotide encoding an anabolic agent fusion protein operably linked to one or more expression control elements are provided.

In some embodiments, a vector is provided that comprises an expression cassette comprising a polynucleotide encoding an anabolic agent fusion protein operably linked to one or more expression control elements, such as a promoter. In some embodiments, the vector is a viral vector, such as but not limited to an adenoviral vector, a lentiviral vector, an adeno-associated viral vector, or a retroviral vector. In some embodiments, the vector is a non-viral vector, e.g., a DNA plasmid. In some embodiments, a polynucleotide is complexed with a delivery vehicle such as a cationic lipid or a cationic polymer. Non-viral vectors are described, for example, in Hardee et al., Genes, 2017, 8:65, doi:10.3390/genes8020065.

### IV. Engineered Cells

In another aspect, the present disclosure provides cells that are engineered to express or overexpress a polynucleotide that encodes an anabolic agent fusion protein. In some embodiments, the engineered cell comprises a heterologous polynucleotide that encodes an anabolic agent fusion protein. In some embodiments, the engineered cell comprises a heterologous expression cassette that comprises a polynucleotide that encodes an anabolic agent fusion protein.

In some embodiments, the cell is a stem cell. In some embodiments, the stem cell is a mesenchymal stem cell (MSC). In some embodiments, the stem cell is a hematopoietic stem cell (HSC).

In some embodiments, an engineered cell expresses a polynucleotide that encodes a fusion protein comprising a mitogenic protein (e.g., PDGF or unmodified or modified FGF) fused to a DSS repeat sequence, as disclosed in Section III above. In some embodiments, the engineered cell expresses a polynucleotide that encodes a fusion protein comprising PDGF (e.g., human PDGF) fused to a DSS repeat sequence having from 2 repeats to 8 repeats (e.g., DSS6).

In some embodiments, an engineered cell comprises a heterologous expression cassette that comprises a polynucleotide that encodes a fusion protein comprising a mitogenic protein (e.g., PDGF or unmodified or modified FGF) fused to a DSS repeat sequence, as disclosed in Section III above. In some embodiments, the engineered cell comprises a heterologous expression cassette that comprises a polynucleotide that encodes a fusion protein comprising PDGF (e.g., human PDGF) fused to a DSS repeat sequence having from 2 repeats to 8 repeats (e.g., DSS6).

For the engineered cells of the present disclosure, the cell can be obtained or derived from any suitable source. In some embodiments, the cell is derived from a fetal source, such as placental amnion, or umbilical cord tissue. In some embodiments, the cell is derived from an adult tissue, such as blood, bone marrow, or adipose tissue. In some embodiments, a stem cell (e.g., HSC or MSC) is a bone marrow-derived cell. In some embodiments, a stem cell (e.g., HSC or MSC) is a cord blood-derived cell. In some embodiments, a stem cell (e.g., HSC or MSC) is a peripheral blood-derived cell. Methods of isolating and generating stem cells are known in the art. See, e.g., Horwitz, 2007, “Sources of Human and Murine Hematopoietic Stem Cells,” Current Protocols in Immunology, 79:A:22A:2:22A.2.1-22A.2.6; Klingemann et al., Transfus Med Hemother, 2008, 35:272-277.

In some embodiments, the cell such as a stem cell (e.g., HSC or MSC) is derived from a human subject. In some embodiments, the cell is derived from a non-human mammal, e.g., a mouse. In some embodiments, the cell is autologous to a subject (e.g., a subject to be administered the engineered cell for the treatment of a bone disease or disorder). In some embodiments, the cell is allogeneic to the subject. In some embodiments, the cell is obtained from a subject that has been administered a chemotherapeutic agent. Methods for obtaining cells such as stem cells are known in the art. For example, stem cells can be obtained through bone marrow aspiration or through apheresis of mobilized peripheral blood cells.

In some embodiments, the engineered cell (e.g., engineered stem cell) overexpresses the anabolic agent fusion protein, as compared to a cell lacking the heterologous polynucleotide. In some embodiments, the engineered cell comprising a heterologous polynucleotide expresses the anabolic agent fusion protein at a level that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, or at least 50-fold higher than a cell lacking the heterologous polynucleotide.

Protein expression can be detected and quantified using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). In some embodiments, protein expression is detected by quantitative mass spectrometry, for example but not limited to, spectral count MS, ion intensities MS, metabolic labeling (e.g., stable-isotope labeling with amino acids in cell culture (SILAC), enzymatic labeling, isotopic labeling (e.g., isotope-coded protein labeling (ICPL) or isotope-coded affinity tags (ICAT)), and isobaric labeling (e.g., tandem mass tag (TMT)).

In some embodiments, the polynucleotide that encodes the anabolic agent fusion protein is introduced into the cell (e.g., stem cell) using a virus or viral vector. In some embodiments, the virus is an adenovirus, lentivirus, adeno-associated virus, or retrovirus. In some embodiments, the virus is a lentivirus. Viruses and viral vectors containing the polynucleotide that encodes an anabolic agent fusion protein can be introduced into the cell by methods known in the art, such as but not limited to, transfection, electroporation, microinjection, transduction, cell fusion, DEAE dextran, calcium phosphate precipitation, or lipofection.

In some embodiments, the polynucleotide that encodes the anabolic agent fusion protein is introduced into the cell (e.g., stem cell) using a non-viral vector. Exemplary non-viral vectors contain the sleeping beauty Tc1-like transposon and a DNA targeting sequence (DTS) facilitating nuclear entry. Non-viral vectors can be introduced into the cell using methods known in the art, such as but not limited to electroporation, insertion of a plasmid encased in liposomes, or microinjection.

In some embodiments, the polynucleotide that encodes the anabolic agent fusion protein is integrated into the genome of the cell (e.g., stem cell), such as into an intron of a safe harbor locus. As used herein, a “safe harbor locus” is a locus in the genome where a polynucleotide may be inserted without causing deleterious effects to the host cell. Examples of safe harbor loci known to exist within mammalian cells may be found within the AAVS 1 gene, the CYBL gene, or the CCR5 gene. In some embodiments, the polynucleotide is integrated into the genome using TALEN-mediated gene targeting. TALEN-mediated gene targeting has been described for stem cells, including human embryonic stem cells (hESCs) and iPSCs (Hockenmeyer et al., Nat Biotechnol 2011, 29: 731-734). Genomic editing with TALENs utilizes a cell's ability to undergo homology directed repair (HDR) following an induced and targeted double-stranded DNA break (DSB). A donor DNA template can be provided to the cell to insert a new transgene or delete DNA sequences at the site of DSB. See, Cheng et al., Genes Cells, 2012, 17:431-8. In some embodiments, a TALEN is designed that targets a safe harbor locus, such as a AAVS 1, CYBL, CCR5, or beta-globin locus.

In some embodiments, clustered regularly interspaced short palindromic repeat (CRISPR) technology is used to integrate a polynucleotide that encodes the anabolic agent fusion protein into the genome of the cell (e.g., stem cell), e.g., into an intron of the safe harbor locus. CRISPR is described, e.g., in Sander et al., Nature Biotechnol 2014, 32:347-355. Briefly, a CRISPR-associated nuclease (Cas, such as Cas9), guided by a single guide RNA (sgRNA) that recognizes a target DNA in the genome through complementary base pairing, binds to the target loci adjacent to a protospacer adjacent motif and generates site-specific double-strand breaks. The double-strand breaks are subsequently repaired either by nonhomologous end-joining (NHEJ) or by HDR upon the existence of a donor template binds to a target loci. The donor template can include a transgene (e.g., a polynucleotide comprising a promoter operably linked to a nucleic acid sequence encoding an anabolic agent fusion protein as disclosed herein). For example, in some embodiments, a cell (e.g., a stem cell such as a HSC) is transformed with a plasmid expressing the Cas9 nuclease, a plasmid encoding an sgRNA that targets the safe harbor (e.g., the AAVS 1 gene, CYBL gene, or CCR5 gene), and a plasmid comprising donor template (e.g., a promoter operably linked to a polynucleotide encoding a PDGF-DSS fusion protein, flanked by homologous arms of the safe harbor gene sequence.

In some embodiments, the engineered cell (e.g., a cell expressing PDGF fused to DSS6) is expanded ex vivo in order to form a population of engineered HSCs. Methods for expanding cells, such as stem cells, are described in the art. See, e.g., Kumar et al., Trends Mol Med, 2017, 23:799-819. In some embodiments, the engineered cells (e.g., stem cells such as HSCs) are expanded in the presence of a suitable expansion medium. For example, in some embodiments, an engineered HSC is expanded in the presence of a hematopoietic stem cell expansion medium, such as Stemline II Hematopoietic Stem Cell Expansion Medium (Sigma). In some embodiments, the expansion occurs in the presence of one or more growth factors or cytokines (e.g., in an expansion medium supplemented with one or more growth factors or cytokines).

In some embodiments, the engineered cell or population of cells is stimulated with one or more growth factors, cytokines, or chemotactic factors. For example, in some embodiments, the engineered cell is a HSC, and the engineered HSC or population of engineered HSCs is stimulated with one or more cytokines or chemotactic factors. Without being bound to a particular theory, it is believed that treating HSCs with a cytokine or chemotactic factor released in the bone marrow can improve the efficiency of HSC homing to bone marrow and subsequent engraftment. Suitable chemotactic factors include, but are not limited to, α-chemokine stromal-derived factor 1 (SDF-1), the bioactive phosphosphingolipids sphingosine-1-phosphate (S1P) and ceramid-1-phosphate (C1P). Suitable cytokines include, but are not limited to, stem cell factor (SCF), IL-3, IL-6, and IL-11. In some embodiments, the engineered HSC or population of engineered HSCs is treated with SCF and/or IL-3.

### V. Pharmaceutical Compositions

In another aspect, pharmaceutical compositions comprising an anabolic agent fusion protein, a nucleic acid (e.g., expression cassette or vector) comprising a polynucleotide encoding an anabolic agent fusion protein, or an engineered cell expressing anabolic agent fusion protein are provided.

In some embodiments, the pharmaceutical compositions are used in a therapeutic method as disclosed herein, e.g., as disclosed in Section VI below. In some embodiments, the pharmaceutical compositions are used in a method of promoting bone growth in a subject in need thereof. In some embodiments, the pharmaceutical compositions are used in a method of treating a disease or disorder that is associated with decreased bone mass. In some embodiments, the pharmaceutical compositions are used in a method of treating a bone injury (e.g., a fracture).

In some embodiments, a pharmaceutical composition comprises an anabolic agent fusion protein, a nucleic acid (e.g., expression cassette or vector) comprising a polynucleotide encoding an anabolic agent fusion protein, or an engineered cell expressing anabolic agent fusion protein as described in Section III or Section IV above and further comprises a pharmaceutically acceptable excipient. Guidance for preparing formulations for use in the present invention is found in, for example, Remington: The Science and Practice of Pharmacy, 21st Edition, Philadelphia, Pa. Lippincott Williams & Wilkins, 2005.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. In some embodiments, the carrier is suitable for intravenous, intramuscular, oral, intraperitoneal, transdermal, topical, or subcutaneous administration. The carrier may be designed to provide a modified or controlled release rate in order to optimize timing of the delivery of the therapeutic agent. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. In some embodiments, the pharmaceutical composition further comprises serum albumin. Other pharmaceutically acceptable carriers and their formulations are well-known and generally described in, for example, Remington: The Science and Practice of Pharmacy, supra. Various pharmaceutically acceptable excipients are well-known in the art and can be found in, for example, Handbook of Pharmaceutical Excipients (5th ed., Ed. Rowe et al., Pharmaceutical Press, Washington, D.C.).

For administration by injection, a fusion protein, nucleic acid, or engineered cell can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, stabilizers and preservatives. In some embodiments, an aqueous solution is used, such as a physiologically compatible buffer such as Hanks's solution, Ringer's solution, or physiological saline buffer. Formulations for injection can be presented in unit dosage form, e.g., in ampules or in multi-dose containers, with an added preservative.

In some embodiments, the pharmaceutical composition is formulated for transdermal administration, for example by a delivery system such as a patch, film, plaster, dressing, or bandage. The delivery system can include any conventional form such as, for example, adhesive matrix, polymeric matrix, reservoir patch, matrix or monolithic-type laminated structure, or other release-rate modifying mechanisms known in the art, and may comprise of one or more backing layers, adhesives, permeation enhancers, an optional rate controlling membrane and a release liner that is removed to expose the adhesives prior to application.

Dosages and concentrations of the anabolic agent fusion protein, nucleic acid encoding the anabolic agent fusion protein, or engineered cells expressing the anabolic agent fusion protein may vary depending on the particular use envisioned (e.g., the therapeutic use and route of administration). Typically the amount of the protein, nucleic acid, or cells administered to a subject is a therapeutically effective amount. In some embodiments, a therapeutically effective amount of an anabolic agent fusion protein, nucleic acid encoding an anabolic agent fusion protein, or engineered cells expressing an anabolic agent fusion protein is an amount that promotes bone growth, e.g., at a site of bone loss or at a fracture site. For example, in some embodiments, a therapeutically effective amount of a fusion protein is a dosage in the range of about 0.01 mg/kg to about 500 mg/kg, or about 0.1 mg/kg to about 200 mg/kg, or about 0.5 mg/kg to about 100 mg/kg, or about 1 mg/kg to about 50 mg/kg. In some embodiments, a therapeutically effective amount of engineered cells is at least about 100, 500, 1,000, 2,500, 5,000, 10,000, 20,000, 50,000, 100,000, 500,000, 1,000,000, 5,000,000, or 10,000,000 cells or more (e.g., per administration). The dosages may be varied depending upon the requirements of the patient, the severity of the condition being treated, and the agent being employed. The dosage amount can also be varied depending upon factors such as the existence, nature, and extent of any adverse side effects that accompany the administration of a particular agent in a particular patient. The determination of the appropriate dosage is well within the skill of the practitioner. Frequently, treatment is initiated with smaller dosages that may be less than the optimum dose of the agent. Thereafter, the dosage is increased by small increments until the optimum effect under the circumstances is reached. For convenience, the total daily dosage may be divided and administered in portions during the day, if desired.

In some embodiments, therapeutic efficacy and optimal dosage is determined or monitored by measuring one or more markers of bone formation. For example, in some embodiments, bone formation is measured by determining the level of the marker serum alkaline phosphatase (ALP), bone alkaline phosphatase (BAP), or procollagen I intact N-terminal propeptide (PINP). Alkaline phosphatase (ALP) is present in a number of tissues including liver, bone, intestine, and placenta. Increased levels of serum ALP can occur due to increased osteoblast activity following accelerated bone growth and in disorders of the skeletal system that involve osteoblast hyperactivity and bone remodeling, such as Paget disease, hyperparathyroidism, rickets and osteomalacia, fractures, and malignant tumors. BAP is a bone-specific isoform of alkaline phosphatase that has been shown to be a sensitive and reliable indicator of bone metabolism. Kress, J Clin Ligand Assay 1998, 21:139-148. Procollagen I intact N-terminal propeptide (PINP) is considered the most sensitive marker of bone formation and it is particularly useful for monitoring bone formation therapies and anti-resorptive therapies.

In some embodiments, therapeutic efficacy and optimal dosage is determined or monitored by measuring bone mineral density. In some embodiments, central dual-energy x-ray absorptiometry (“central DXA”) is used for measuring bone mineral density. Bone mineral density can be measured in a variety of places of the skeleton, such as the spine or the hip. In some embodiments, Generally, a BMD test measures the amount of bone mineral in one or more bones, such as the spine, hip, or wrist. Bone density can be calculated by several different methods, including the T-score and Z-score. The T-score is the number of standard deviations above or below the mean for a healthy 30 year-old adult of the same sex at the patient. The Z-score is the number of standard deviations above or below the mean for the patient's age and sex. In some embodiments, an improvement in a patient's T-score or Z-score (e.g., from a baseline level determined before the onset of treatment) indicates bone growth.

### VI. Methods of Treatment

In another aspect, therapeutic methods comprising the use of an anabolic agent fusion protein, a nucleic acid (e.g., expression cassette or vector) comprising a polynucleotide encoding an anabolic agent fusion protein, an engineered cell expressing anabolic agent fusion protein, or a pharmaceutical composition comprising an anabolic agent fusion protein or a nucleic acid comprising a polynucleotide encoding an anabolic agent fusion protein are provided. In some embodiments, the therapeutic methods relate to promoting bone growth in a subject in need thereof. In some embodiments, the therapeutic methods relate to treating a disease or disorder that is associated with decreased bone mass. In some embodiments, the therapeutic methods relate to treating a bone injury (e.g., a fracture).

In some embodiments, a subject to be treated according to a method disclosed herein is a human subject. In some embodiments, the subject is an adult. In some embodiments, the subject is a juvenile.

### Fracture Repair

In one aspect, methods of treating a fracture are provided. In another aspect, methods of accelerating fracture healing are provided. In some embodiments, the method comprises administering to a subject having a fracture an anabolic agent fusion protein, a nucleic acid comprising a polynucleotide sequence encoding the anabolic agent fusion protein, a hematopoietic stem cell expressing the anabolic agent fusion protein, or a pharmaceutical composition comprising the anabolic agent fusion protein, wherein the anabolic agent fusion protein comprises PDGF or unmodified or modified FGF fused to a DSS repeat sequence. In some embodiments, the anabolic agent fusion protein is a protein as disclosed in Section III above. In some embodiments, the pharmaceutical composition comprising the anabolic agent fusion protein is a composition as disclosed in Section IV above. In some embodiments, the anabolic agent fusion protein is PDGF-BB fused to a DSS6 repeat sequence.

In some embodiments, the fracture is a fracture of a tibia, fibula, femur, radius, ulna, tarsal, metatarsal, carpal, metacarpal, vertebra, clavicle, or pelvis. In some embodiments, the fracture is a fracture of a long bone. In some embodiments, the fracture is a fracture of a vertebra. In some embodiments, the fracture is a fracture from a military wound or gunshot wound (e.g., a fracture resulting from a blast injury such as from an improvised explosive device or shrapnel). In some embodiments, the fracture is a result of a medical condition or disease. For example, in some embodiments, the fracture is a result of a disease or condition such as osteogenesis, brittle bone disease (osteogenesis imperfecta), cancer, metabolic bone disease.

In some embodiments, the fracture is a delayed union fracture, an established nonunion fracture, or a simple fracture. The definitions of “fracture union,” “delayed fracture union,” “nonunion,” and “simple fracture” are well-known in the art. See, e.g., Marsh, Clin Orthop Relat Res, 1998, S22-30. As used herein, a “delayed union fracture” refers to a fracture that take longer than typical to heal (e.g., as compared to the typical fracture healing time period for that injury, e.g., for a given bone in a given species). In some embodiments, a delayed union fracture is a fracture that takes at least twice as long to heal as is typical for that injury. As used herein, a “nonunion” refers to a fracture that fails to heal. It is within the ordinary level of skill in the art to determine whether an injury exhibits a delay in fracture union or exhibits nonunion. As used herein, a “simple fracture” refers to a fracture of the bone only that does not penetrate the skin.

In some embodiments, administration of an anabolic agent fusion protein as disclosed herein accelerates delayed fracture healing. For example, in some embodiments, administration of the anabolic agent fusion protein accelerates delayed fracture healing by at least 10%, at least 20%, at least 30%, at least 40%, at least 50%, at least 60%, at least 70%, at least 80%, at least 90% or more as compared to a control value (e.g., as measured by time to heal for a fracture of the same bone in a subject not treated with the anabolic agent fusion protein).

### Methods of Increasing Bone Growth

In another aspect, methods of increasing bone growth are provided. In some embodiments, the method comprises administering to a subject in need thereof an anabolic agent fusion protein, a nucleic acid comprising a polynucleotide sequence encoding the anabolic agent fusion protein, a hematopoietic stem cell expressing the anabolic agent fusion protein, or a pharmaceutical composition comprising the anabolic agent fusion protein, wherein the anabolic agent fusion protein comprises PDGF or unmodified or modified FGF fused to a DSS repeat sequence. In some embodiments, the composition (e.g., fusion protein, nucleic acid, hematopoietic stem cell, or pharmaceutical composition) is a composition as disclosed in Section III or Section IV above. In some embodiments, the anabolic agent fusion protein is PDGF-BB fused to a DSS6 repeat sequence.

In some embodiments, a method of increasing bone growth comprises administering to the subject a therapeutically effective amount of engineered cells that express an anabolic agent fusion protein or a pharmaceutical composition comprising the engineered cells, wherein the anabolic agent fusion protein is a fusion protein as disclosed in Section III above (e.g., a fusion protein comprising PDGF or unmodified or modified FGF fused to a DSS repeat sequence).

In some embodiments, the method comprising administering to the subject an anabolic agent fusion protein (e.g., as disclosed in Section III above, e.g., a fusion protein that comprises PDGF or unmodified or modified FGF fused to a DSS repeat sequence). In some embodiments, the anabolic agent fusion protein is locally administered to a site of bone loss or bone injury.

In some embodiments, the methods of increasing bone growth are used in the treatment of a subject having low bone mass or having a disease, disorder, or condition characterized by low bone mass or loss of bone mass. Diseases, disorders, and conditions characterized by low bone mass or loss of bone mass include, but are not limited to, osteoporosis (e.g., primary osteoporosis and idiopathic primary osteoporosis); genetic disorders such as cystic fibrosis, Ehlers-Danlos syndrome, glycogen storage diseases, Gaucher's Disease, hemochromatosis, homocystinuria, hypophosphatasia, idiopathic hypercalciuria, Marfan syndrome, Menkes Steely Hair syndrome, osteogenesis imperfecta, porphyria, Riley-Day syndrome, and Werner's syndrome; hypogonadal states such as androgen insensitivity, anorexia nervosa, athletic amenorrhea, hyperprolactinemia, panhypopituitarism, premature ovarian failure, Turner's syndrome, Kleinfelter's syndrome; endocrine disorders such as acromegaly, adrenal insufficiency, Cushing's syndrome, diabetes mellitus (type 1), hyperparathyroidism, hyperthyroidism, and thyrotoxicosis; gastrointestinal diseases such as gastrectomy, inflammatory bowel disease, malabsorption, celiac disease, primary biliary cirrhosis, Crohn's disease, and ulcerative colitis; hematologic disorders such as hemophilia, leukemias, lymphomas, multiple myeloma, sickle cell disease, systemic mastocytosis, and thalassemia; rheumatic and auto-immune diseases such as ankylosing spondylitis, Graves' disease, juvenile rheumatoid arthritis, lupus, and rheumatoid arthritis; and other conditions such as alcoholism, amyloidosis, arthritis, bone cancer, chronic metabolic acidosis, congestive heart failure, depression, emphysema, end stage renal disease, epilepsy, idiopathic scoliosis, immobilization, multiple sclerosis, muscular dystrophy, osteoarthritis, osteomalacia, osteomyelitis, Paget's disease, polyostotic fibrous dysplasia, pregnancy-associated osteoporosis, post-transplant bone disease, sarcoidosis, and zero gravity. In some embodiments, the subject has medication-associated low bone mass or loss of bone mass, such as low bone mass induced by anticoagulants (e.g., heparin), anticonvulsants, cyclosporine A, tacrolimus, cancer chemotherapeutic drugs, corticosteroids, glucocorticoids, ACTH, gonadotropin-releasing hormone agonists, immunosuppressants, lithium, methotrexate, parenteral nutrition, or thyroxine.

In some embodiments, the subject has low bone density. Methods for measuring bone density are known in the art. In some embodiments, a bone mineral density (BMD) test is used. Generally, a BMD test measures the amount of bone mineral in one or more bones, such as the spine, hip, or wrist. In some embodiments, central dual-energy x-ray absorptiometry (“central DXA”) is used for measuring BMD. For determining a BMD score, typically a subject's results are compared to the ideal or peak bone mineral density of a healthy 30-year-old adult and are reported as a “T-score.”

In some embodiments, a subject to be treated has a bone density score (T-score) that is between 1 and 2.5 SD below the young adult mean (−1 to −2.5 SD). In some embodiments, a subject to be treated has a bone density score (T-score) that is 2.5 SD or more below the young adult mean (−2.5 SD or lower). In some embodiments, a T-score that is −2.5 SD or lower is indicative of osteoporosis. In some embodiments, a subject to be treated has a bone density score (T-score) that is more than 2.5 SD below the young adult mean, and has had one or more osteoporotic fractures. In some embodiments, a T-score that is −2.5 SD or lower in combination with a history of one or more fractures is indicative of severe osteoporosis.

In some embodiments, a subject to be treated has osteoporosis. In some embodiments, a subject to be treated has severe osteoporosis. In some embodiments, the subject has primary osteoporosis, which as used herein refers to a loss of bone mass unrelated to any other underlying disease or illness. In some embodiments, the primary osteoporosis is Type I osteoporosis. In some embodiments, the primary osteoporosis is Type II osteoporosis. In some embodiments, the primary osteoporosis is idiopathic osteoporosis. In some embodiments, a subject to be treated has bone loss arising from a secondary condition, e.g., from a disease, disorder, or condition as disclosed above.

In some embodiments, a therapeutic method as disclosed herein (e.g., a method of treating a fracture or a method of increasing bone growth) comprises locally administering a composition as disclosed herein, e.g., an anabolic agent fusion protein, nucleic acid comprising a polynucleotide sequence encoding the anabolic agent fusion protein, hematopoietic stem cell expressing the anabolic agent fusion protein, or pharmaceutical composition comprising an anabolic agent fusion protein. In some embodiments, the composition is administered locally to a bone or at a site of low bone mass. In some embodiments, the composition is administered systemically. In some embodiments, the anabolic agent fusion protein is administered by injection. In some embodiments, the anabolic agent fusion protein is administered by micropump, or as performed by other similar devices such as insulin pumps. In some embodiments, the anabolic agent fusion protein is administered transdermally, e.g., using a transdermal patch.

In some embodiments, a single dose of the anabolic agent fusion protein is administered to the subject. In some embodiments, multiple doses of the anabolic agent fusion protein are administered. In embodiments wherein multiple doses of the anabolic agent fusion protein are administered, the administration of the doses can be spaced out over the course of days, weeks, or months. In some embodiments, multiple doses are administered over the course of about 1 day, 2 days, 3 days, 4 days, 5 days, 6 days, 7 days, 8 days, 9 days, 10 days, 11 days, 12 days, 13 days, 14 days, 15 days, 20 days, or 25 days. In some embodiments, multiple doses are administered over the course of about 1 week, 2 weeks, 3 weeks, 4 weeks, 5 weeks, 6 weeks, 7 weeks, 8 weeks, 9 weeks, 10 weeks, 11 weeks, 12 weeks, or longer. In some embodiments, multiple doses are administered over the course of about 1 month, 2 months, 3 months, 4 months, 5 months, 6 months, 7 months, 8 months, 9 months, 10 months, 11 months, 12 months, or longer. In some embodiments, the anabolic agent fusion protein is administered to the subject for a predetermined time, an indefinite time, or until an endpoint is reached. In some embodiments, treatment is continued on a continuous daily or weekly basis for at least two to three months, six months, one year, or longer. In some embodiments, treatment is for at least 30 days, at least 60 days, at least 90 days, at least 120 days, at least 150 days, or at least 180 days. In some embodiments, treatment is continued for at least 6 months, at least 7 months, at least 8 months, at least 9 months, at least 10 months, at least 11 months, or at least one year. In some embodiments, treatment is continued for the rest of the subject's life or until administration is no longer effective to provide meaningful therapeutic benefit.

### VII. Kits

In still another aspect, kits comprising an anabolic agent fusion protein, a nucleic acid (e.g., expression cassette or vector) comprising a polynucleotide encoding an anabolic agent fusion protein, an engineered cell expressing anabolic agent fusion protein, or a pharmaceutical composition as disclosed herein are provided. In some embodiments, the kit comprises an anabolic agent fusion protein as disclosed in Section III above. In some embodiments, the kit comprises a nucleic acid (e.g., expression cassette or vector) comprising a polynucleotide encoding an anabolic agent fusion protein as disclosed in Section III above. In some embodiments, the kit comprises an engineered cell expressing anabolic agent fusion protein as disclosed in Section IV above. In some embodiments, the kit comprises a pharmaceutical composition as disclosed in Section V above.

In some embodiments, the kits are used in a therapeutic method as disclosed herein, e.g., as disclosed in Section VI above. In some embodiments, the kits are used in a method of promoting bone growth in a subject in need thereof. In some embodiments, the kits are used in a method of treating a disease or disorder that is associated with decreased bone mass (e.g., osteoporosis). In some embodiments, the kits are used in a method of treating a bone injury (e.g., a fracture).

In some embodiments, the kit further comprises instructional materials containing directions (i.e., protocols) for the practice of the methods of this disclosure (e.g., instructions for using the kit for treating fracture repair or promoting bone growth). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD-ROM), and the like. Such media may include addresses to internet sites that provide such instructional materials.

### VIII. Examples

The following examples are offered to illustrate, but not to limit, the claimed invention.

### Example 1

### Generation and Characterization of PDGF-DSS6 Fusion Protein

It has been observed that growth factors that are purely mitogenic such as FGF2 and PDGF are exceedingly effective as anabolic agents. Neither FGF2 nor PDGF increases bone formation at the periosteum, only bone formation in marrow, and PDGF has no ability in vitro to promote bone formation, only cell proliferation. These observations demonstrate that the marrow space has a unique molecular environment to promote bone formation once primitive mesenchymal cells (precursors of osteoblasts) are recruited and expanded by PDGF. As such, mitogenic agents introduced into the marrow space become osteogenic. In support of this concept, the introduction of mesenchymal stem cells overexpressing PDGF produce trabecular bone in the diaphyseal marrow space where trabecular bone does not normally occur. See, Chen et al., PNAS, 2015, 112:E3893-3900.

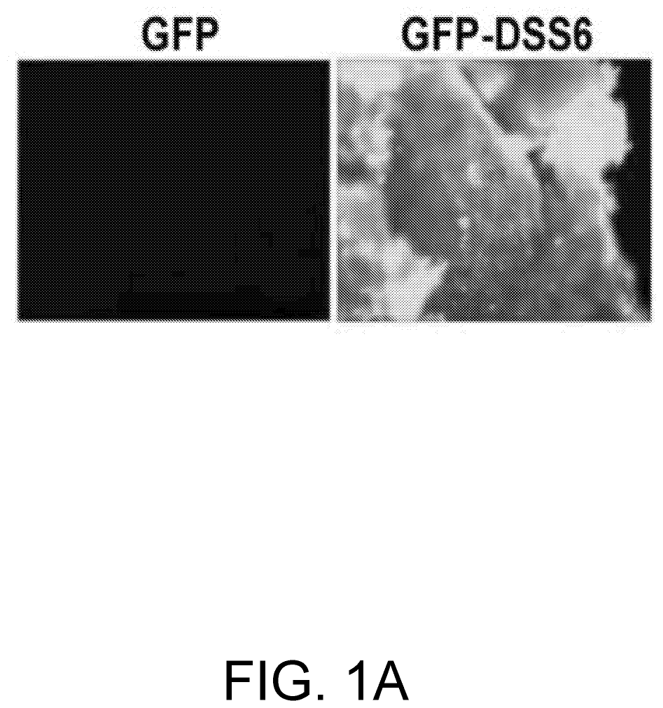

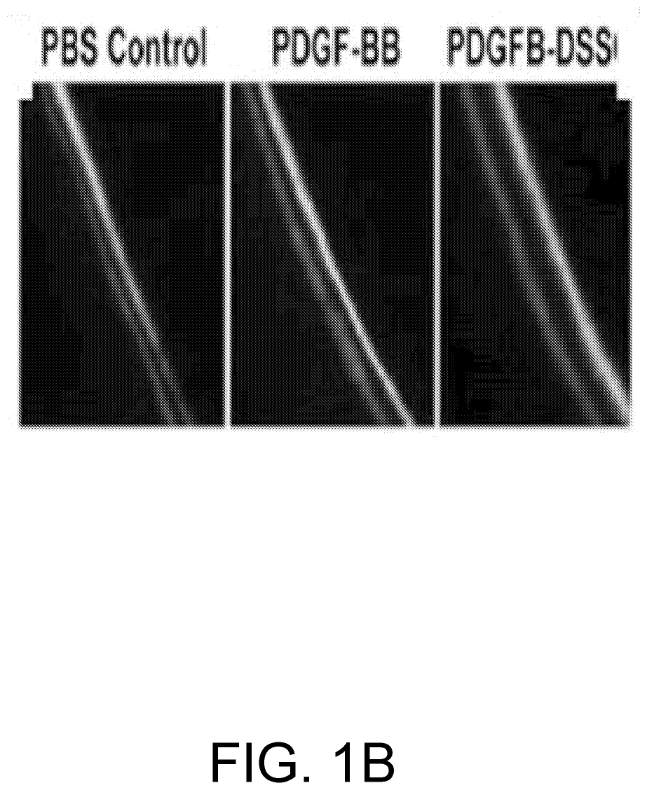

However, PDGF receptors are expressed by multiple tissues. Furthermore, PDGF-B is a mitogen that can promote proliferation of multiple types of cells; accordingly high-level serum PDGF-B has potential risks. To prevent unintended effects that may occur in clinical trials, it is desirable to confine PDGF to bone surface. Therefore, a targeting strategy was developed by fusing PDGF to a bone surface binding peptide comprising multiple repeats of Asp-Ser-Ser (DSS). Specifically, DSS6, a six repeating sequence of AspSerSer, was chosen as a fusion partner of PDGF-B to constrain PDGF-B to bone surface instead of circulation. DSS6 has been reported to bind to calcium phosphate compounds of the bone surface with high affinity. We first sought to confirm that DSS6 could efficiently bind and localize DSS6 fusion protein at the bone surface in vitro. Indeed, incubation of GFP-DSS6 fusion protein with bone chips produced an intense green fluorescence as compared with GFP alone, demonstrating that DSS6 fusion protein can target and bind to the bone (FIG. 1A).

The ability of a PDGFB-DSS6 fusion protein to promote bone formation in vivo was compared to PDGF-B without the calcium-binding peptide. Mice were injected with PDGF-B or with PDGFB-DSS6 twice a week for 3 weeks, then the mineral apposition rate was examined by two-color dynamic histomorphometry. FIG. 1B shows dynamic histomorphometry of the midshaft femoral endosteum using Xylenol (red) and Calcein (green) labels in PBS, 1 mg/kg PDGF-B or 1 mg/kg PDGF-B/DSS5 animals. As shown in FIG. 1B, it was surprisingly found that the intra-label width of bone formed in mice administered PDGFB-DSS6 was much bigger than that of mice administered PGDG-B. This intra-label width reflects the synthetic activity of individual osteoblasts, and suggests that the PDGF-DSS6 fusion protein is able to increase osteoblast number and/or osteoblast activity.

### Example 2

### Generation of Hematopoietic Stem Cells Expressing PDGF-DSS6

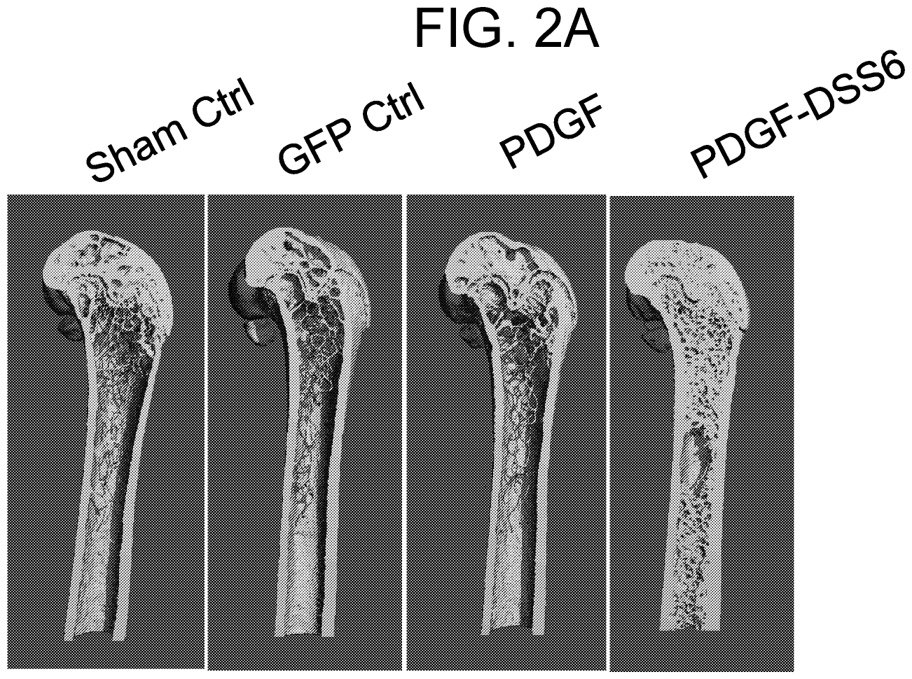

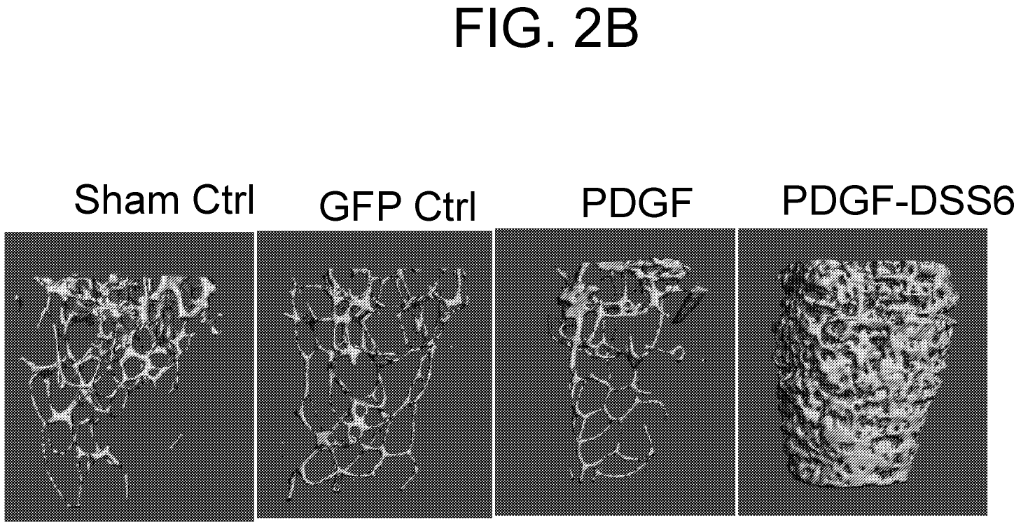

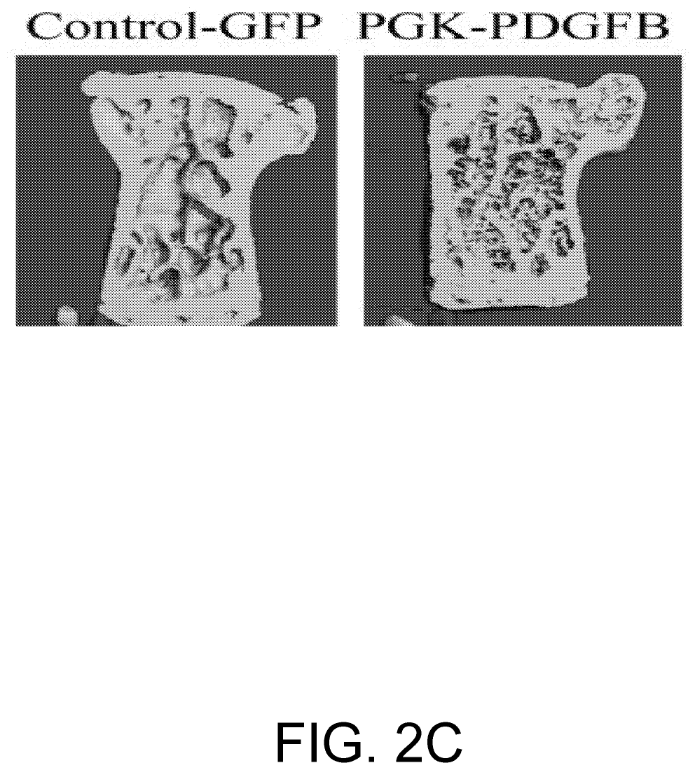

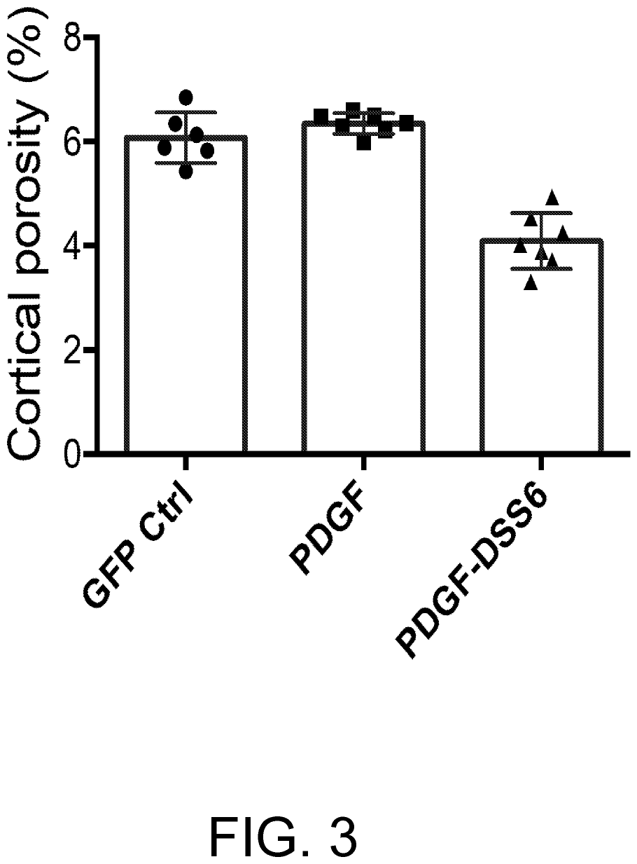

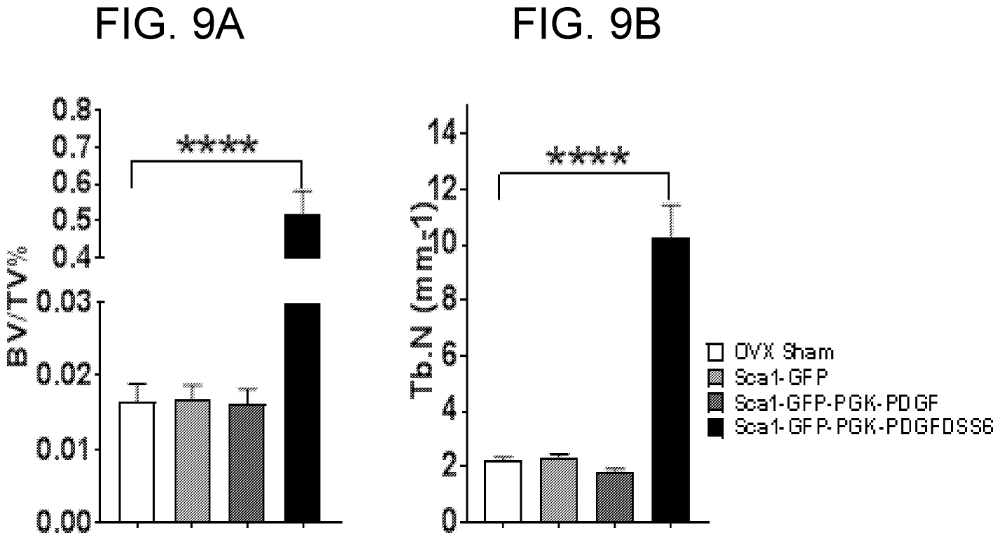

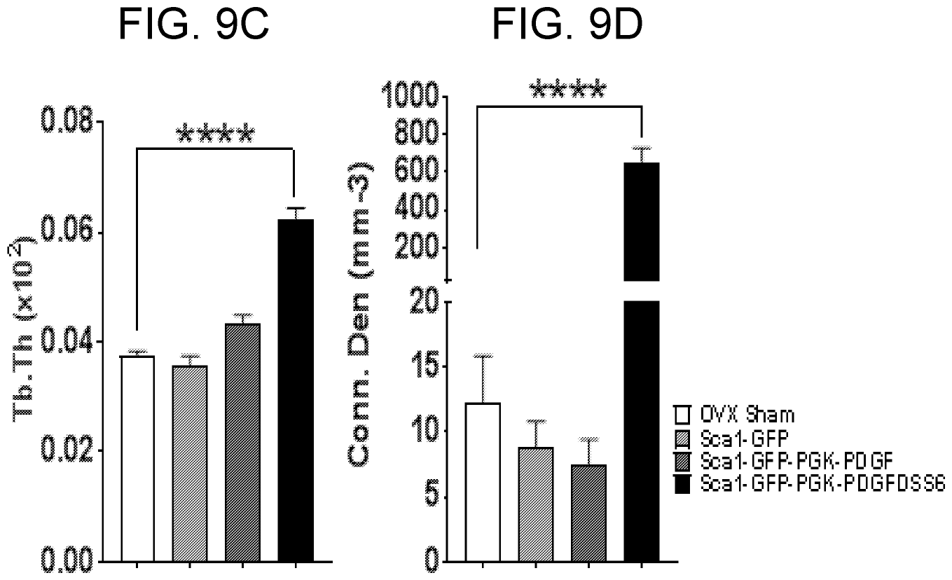

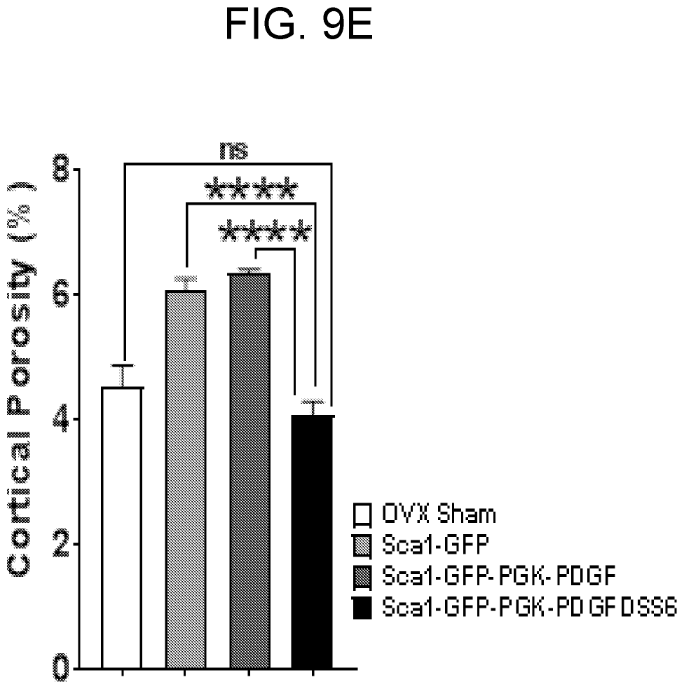

A bone marrow transplant was performed in an OVX osteoporosis mouse model with hematopoietic stem cells engineered to produce PDGF or PDGF-DSS6. The Lenti PGK-PDGF-DSS6 vector was used for this experiment. The expression of PDGF-DSS6 fusion gene was driven by the PGK (Phosphoglycerate kinase) promoter. The lentiviral vector was packaged in 293T cells by using standard protocol. Sca1⁺ cells (hematopoietic stem/progenitors) were enriched from mouse bone marrow. After culturing for 2 days, Sca1⁺ cells were transduced with Lenti PGK-PDGF-DSS6 vector at an MOI (multiple of infection) of 4. One day after viral transduction, Sca1⁺ cells engineered with PDGF-DSS6 were transplanted to irradiated mice. Strikingly, we found that even with low-level engraftment of transduced HSCs (3-5%), significant efficacy of PDGF-DSS6, but not wild type PDGF, was achieved (FIG. 2A-FIG. 2B). As shown in FIG. 2A-FIG. 2B, hematopoietic stem cells expressing PDGF-DSS6 increased microCT evaluated bone density more than 10-fold as compared to hematopoietic stem cells expressing PDGF. Without being bound to a particular theory, the significantly higher level of bone growth achieved using PDGF-DSS6 may be due to increased stability and high-level local concentration of PDGF-DSS6 at the bone surface. In addition, PDGFB-DSS6 also decreased the OVX-induced cortical porosity (FIG. 3). Transduction of Sca-1⁺ cells with PDGF-B into mice also resulted in a marked anabolic effect on the tail vertebra (FIG. 2C), demonstrating that PDGF is a potent stimulator of bone formation in both red and yellow marrow. Further, BV/TV percent was more than 10 fold increased in the Sca 1 PGK PDGF DSS6 compared to the Sca 1 PDGF or the Sca 1 GFP. Trabecular number, trabecular thickness and trabecular cut activity or all much greater in the Sca 1 PGK PDGF-DSS6 that in the Sca 1 PGK PDGF or Sca 1 PGK GFP (FIGS. 9A-9E). Connectivity density was more than 10 fold higher than and the Sca1 PDGF group. Thus our therapy had a strong ability to cause de novo bone formation; i.e., trabecular bone formation where there was no bone before. We also measured cortical porosity by microCT and found a highly significant decrease suggesting that our Femur length was unchanged stem cell gene therapy had no action on endochondral ossification (FIGS. 9A-9E).

### Example 3

### Administration of PDGF-DSS6 Protein

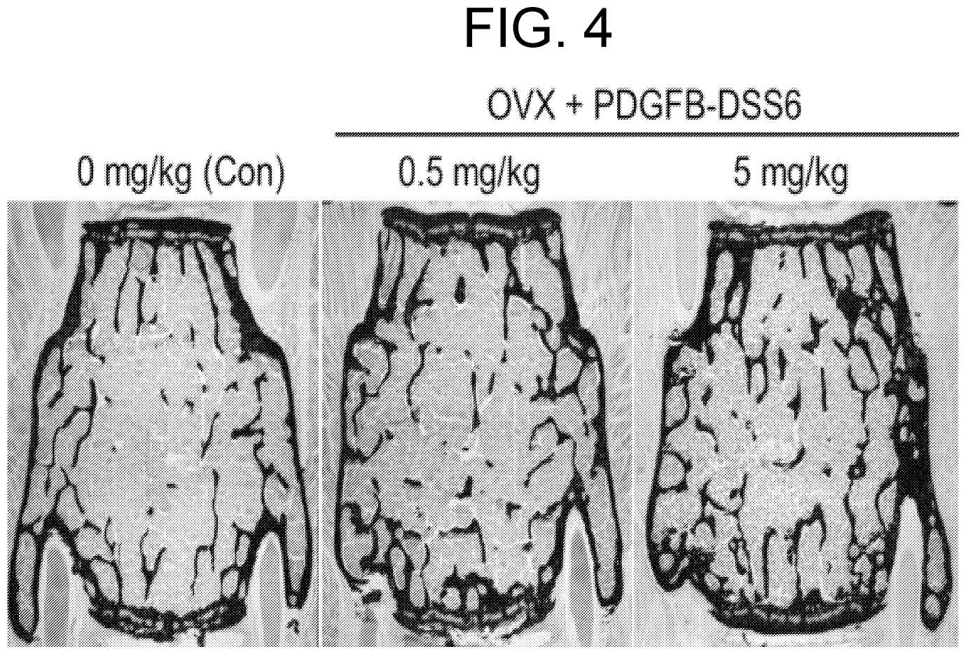

Next, the effects of administration of PDGF-DSS6 protein were tested. At 1 month after ovariectomy, mice were dosed with PBS or PDGFB-DSS6 (0.5 mg/kg or 5 mg/kg) intravenously three times per week for 4 weeks. Intravenous injection of PDGF-DSS6 not only increased bone formation in femurs (data not shown), but also in lumbar vertebrae (FIG. 4) in a dose-dependent manner. FIG. 4 shows that there was an approximately 30% increase in newly deposited bone in the vertebra after 4 weeks of intravenous therapy with PDGF-DSS6 protein without any evidence of abnormal bone formation or osteomalacia. These results demonstrate that injection of PDGF-DSS6 protein is efficacious for promoting bone growth.

For identifying an optimal dose of PDGF-DSS6, the OVX osteoporosis mouse model can be used. An ALZET osmotic pump is used to administer PDGF-DSS6 protein for 4 weeks of continuous delivery, which is expected to increase the concentration of PDGF-DSS6 on the marrow bone surfaces without increasing serum level of total PDGF. PDGF-DSS6 is delivered continuously for 4 weeks by subcutaneously embedded osmotic pumps at one of three doses: (1) 0 mg/kg (negative control, PBS only), (2) 2 mg/kg (low dose), (3) 5 mg/kg (medium dose), and (4)15 mg/kg (high dose). Animals (6-8 week-old female C57BL/6 mice, Jackson Laboratories) are randomly assigned to each group. For determining therapeutic efficacy, serum PDGF-DSS6 is measured every 2 weeks. To distinguish human PDGFB from mouse Pdgfb, the administered PDGFB-DSS6 protein has a FLAG tag that allows for quantitating human PDGFB using FLAG-tag ELISA Kits. To assess the bone apposition rate, xylenol orange and calcein green, administered subcutaneously at 90 mg/kg and 10 mg/kg, respectively, provide easily identified and differentiated bands in newly deposited bone within periosteal and endosteal calluses, intercortical gaps, and screw holes. Bone formation markers like serum ALP (alkaline phosphatase) are measured every 2 weeks. At 0 or 1 month after treatment, mice are evaluated for bone mineral density and microstructure by microCT, bone strength, and bone histomorphometry analysis. Experimental details are described, for example, in Chen et al., PNAS, 2016, 112:E3893-3900; Meng et al., PloS one, 2012, 7:e37569, Su et al., PloS one, 2013, 8:e64496, and Lau et al., Bone, 2013, 53:369-381. Toxicity of PDGF-DSS6 treatment can also be evaluated on major organs by histology and transcriptome analysis.

### Example 4

### Study Design

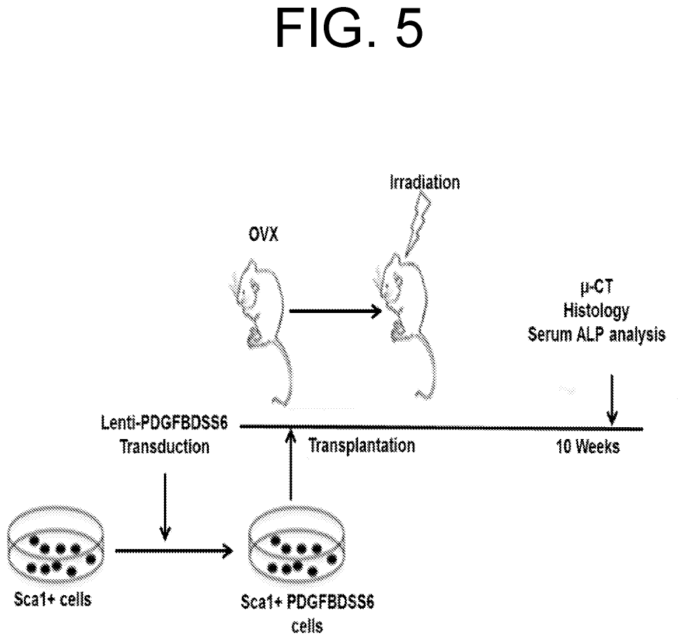

The goal of this study was to study in OVX mice the effects of our stem cell gene therapy, Sca 1 PDGF with the new transgene for bone specific targeting: Sca 1 PDGF-DSS6. The primary end point was microCT to evaluate the amount and distribution of the new bone formed in response to our stem cell gene therapy. Secondary endpoints include x-rays of long bones, Von Kossa stain histology, and serum alkaline phosphatase. Animal groups of 7 mice each were: OVX sham, OVX GFP (untreated), OVX Sca 1 PDGF and OVX Sca 1 PDGF-DSS6. Study design as shown in FIG. 5. The major issue was whether PDGF-DSS6 was superior to PDGF alone in anabolic action.

### Example 5

### Bone Marrow Transplantation

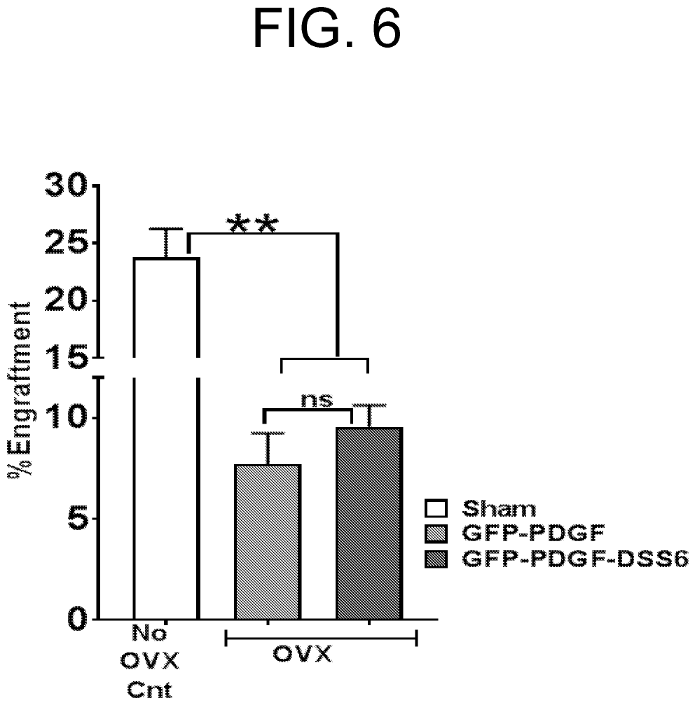

After total body irradiation each mouse in the stem cell therapy group was injected with 1 million Sca 1 cells IV. To evaluate the level of engraftment of the Sca 1 cells serum GFP was measured 1 and 2 months after delivery of the Sca 1 cells. In our Sca 1 GFP OVX control engraftment was about 18% which is similar to what we have seen in the past. However in the two OVX groups (PDGF and PDGF-DSS 6) engraftment was only 8% (FIG. 6). These results raise the possibility that Sca 1 cells engineered to overexpress PDGF have a decreased ability to engraft in the OVX animal.

### Example 6

### Serum ALP at 10 Weeks Post Therapy

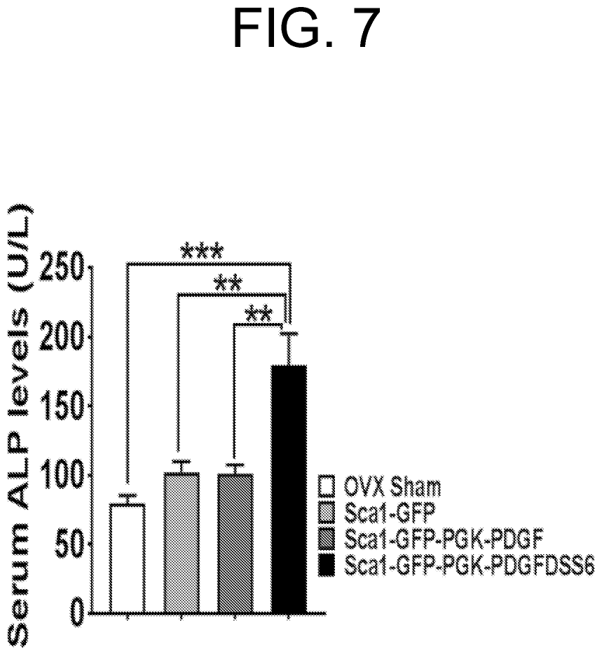

At 10 weeks after the beginning of stem cell gene therapy serum ALP measurements reveal that compared to the OVX sham group there was no significant increase in the Sca1 GFP group or the Sca 1 GFP PGK PDGF group; however, the serum ALP was more than doubled in the Sca 1 GFP PGK PDGF DSS 6 group (FIG. 7).

### Example 7

### X-Ray of the Femur Long Bones

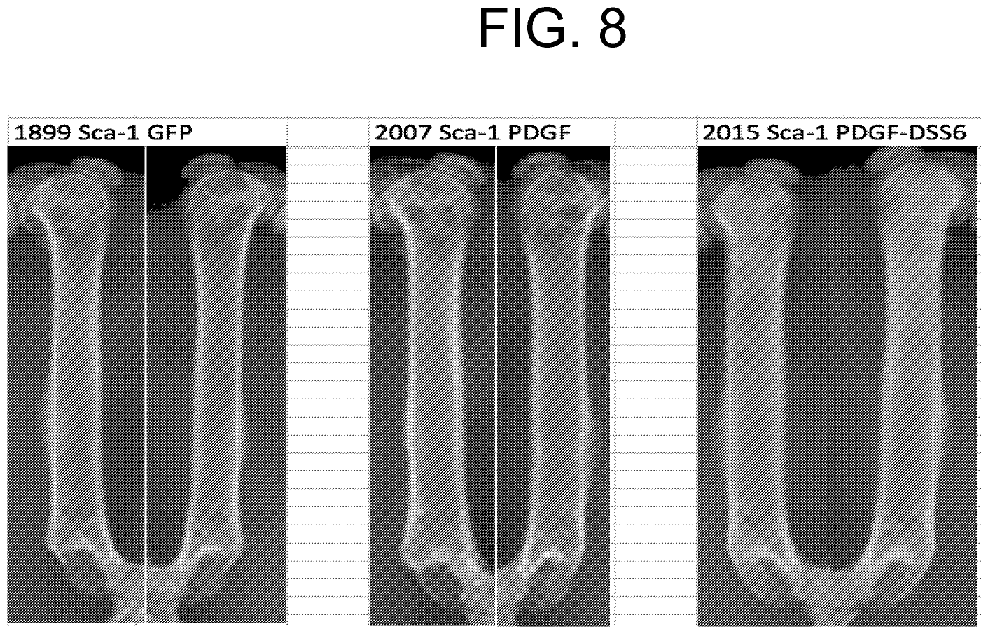

X-rays obtained after 10 weeks of therapy show increased trabecular bone particularly in the metaphysis and femoral trochanter and also a suggestion of cortical thickening in the Sca 1 PGK PDGF DSS6 compared to the Sca 1 PGK PDGF and Sca 1 PGK GFP (FIG. 8).

### Example 8

### Bone Strength Analysis

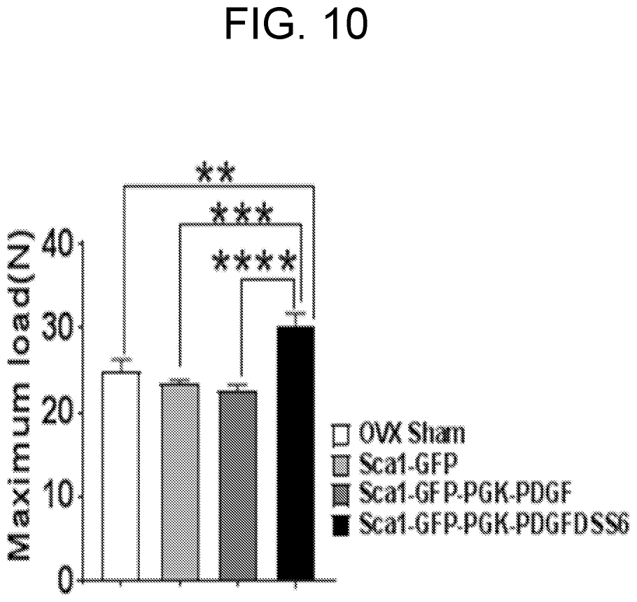

Bone strength was measured by the 3-point bending of the right femurs. The Sca 1 PDGF DSS 6 group showed a significant increase in bone strength compared to the other 3 groups including the OVX sham. Of interest, we also observed that the cortical porosity was decreased from 6% to 4% after PDGF-DSS6 treatment (FIG. 10).

### Example 9

### Bone Histology

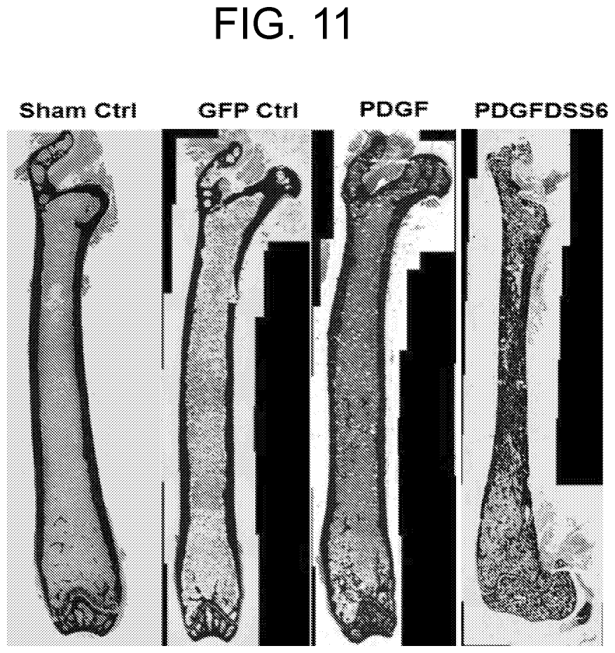

The von Kossa stain is commonly used to quantify mineralization; we then decided to investigate mineralization levels after PDGFB-DSS6 treatment. Utilizing a von Kossa stain to label calcium and hematoxylin and eosin dyes to label the chromatin and cellular structures, we can see a difference in the matrices of the bone (FIG. 11). As shown in the FIG. 11, PDGFB-DSS6 based gene therapy promotes bone regeneration in the OVX osteoporosis mouse model. These data suggest that PDGFB-based therapy is clinically relevant to treat patients with severe bone loss.

### Example 10

### Materials and Methods

Animal study

Five-week-old female C57BL/6J mice were purchased from the Jackson laboratory. All experimental protocols were approved by the Institutional Animal Care and Use Committee at Loma Linda University and the Animal Care and Use Review Office of the United States Department of the Army. In conducting research using animals, the investigators adhered to the Animal Welfare Act Regulations and other Federal statutes relating to animals and experiments involving animals and the principles set forth in the current version of the Guide for Care and Use of Laboratory Animals, National Research Council.

OVX Surgery

OVX surgery was done on 2 month old C57BL/6J female mice. Mice were anesthetized by an intraperitoneal injection of 105 μg/kg ketamine and 21 μg/kg xylazine (in a total of ˜0.1 ml volume). Body temperature is maintained by a 37° C. recirculating-water heating pad. The back and sides of the mice were shaved and cleaned with 70% ethanol and Betadine. Under aseptic conditions, the pair of ovaries was removed from the mice by dorsal incision into the region between the dorsal hump and the base of the tail. Removal of the fimbrial end of the fallopian tube was done to ensure completeness of the ovariectomy. The muscle incision is closed with 6-0 silk sutures, and skin incisions closed with 3-0 silk sutures. Post-operative analgesic (0.060 mg/kg in 0.05 ml buprenorphine, subcutaneously) was administered for each mouse. After surgery, animals were treated for two days, twice a day with buprenorphine, and monitored closely thereafter. The animals were observed during recovery until alert and mobile. The surgical procedure for control, sham-operated mice is the same except that the ovaries are not excised.

BM Sca-1⁺ Cell Isolation and Transplantation

Bone marrow Scar cell isolation was performed as previously described. Briefly, bone marrow cells were harvested from femurs and tibias, and Sca1⁺ cells were purified using Sca1 MACS magnetic beads (MiltenyiBiotec, cat no 130-106-641). Before viral transduction, cells were cultured for 48 hr. in Iscove's modified Dulbecco's medium (IMDM, Invitrogen) containing 10% FBS (Invitrogen) and 100 ng/mL each of human TPO, mouse SCF, human FL, human IL-3, and human G-CSF.

Irradiation/Transplantation

OVX mice were irradiated with a 60Co source (Eldorado model, Atomic Energy of Canada) at a single dose of 6 Gy (0.543 Gy/min). When the mice are around 3-4 months old, they received 6 Gy irradiation from a 60Co source machine in the Department of Radiation Medicine. Twenty-four hours later, 1.0×106 lentiviral transduced Sca1⁺ cells were resuspended in 200 μL IMDM and transplanted into each recipient mouse via tail vein injection. The mice are restrained in the mouse restrainer during tail vein injection, and then properly returned to their cages.

MicroCT Analysis

A μCT analysis of the femoral bone was performed using a Scanco VivaCT 40 instrument (Scanco Medical). Femurs were scanned at an isotropic voxel size of 10.4 μm3, and energies of 55 keV and 70 keV were used to scan the metaphysis and midshaft, respectively. For metaphyseal scans, the region of interest was a set distance proximal to the condylar growth plate after the bone was normalized for variations in length. Midshaft scans were also normalized for any variations in bone length. Trabecular bone was segmented at a density of greater than 220 mg/cm3 and the cortical bone segmented at a density of greater than 260 mg/cm3. Cortical porosity parameters were determined from the true 3D indices as calculated from a two contour examination of the femoral midshaft cortex. The cortical porosity was calculated as 1-BV/TV (midshaft).

Bone Strength Analysis

The mechanical strength of the femurs was evaluated at midshaft by the three-point bending test, using an lnstron DynaMight 8841 servohydraulic tester (Instron). Bones were stored frozen in saline-soaked gauze, and thawed and rehydrated in saline before testing. The femur was positioned on the tester with the anterior aspect upwards on supports that were 7 mm apart. The bone was preloaded to 1 N at the midshaft and then loaded to failure using a blade excursion rate of 5.0 mm/s.

Serum Alkaline Phosphatase Measurement

Serum ALP activity was measured by QuantiChrom ALP kit (BioAssay Systems).

Bone Histology

Mouse femurs from OVX non treated and treated were fixed in 1% paraformaldehyde (PFA) containing 0.1% picric acid overnight at 4° C., and frozen sections were obtained as previously described. Briefly, undecalcified femurs were embedded in SCEM embedding medium (Section Lab). After trimming, the sample surface was covered with the Cryofilm type 2C adhesive film (Section Lab) and 5-μm-thick sections were cut using Cryostat (Leica CM3050S). The sections were used for von Kossa staining of mineralized bone matrix. For von Kossa staining, sections were first incubated with 1% silver nitrate solution for 45 min, then rinsed thoroughly in distilled water, and subsequently incubated with 5% (wt/vol) thiosulfate solution for 5 min. After washes, sections were counterstained with H&E. All images were captured using an Olympus BX51 microscope system (Olympus).

GFP DSS 6 Binding to Bone Chips

We choose DSS6, a six repeating sequence of AspSerSer, as a fusion partner of PDGFB to constrain PDGFB to bone surface instead of circulation. DSS6 has been reported to bind to calcium phosphate compounds of the bone surface with high affinity. We first sought to confirm that DSS6 could efficiently bind and localize DSS6 fusion protein at the bone surface in vitro. Bone chips were incubated with green fluorescent protein (GFP) or GFP-DSS6 (50 ng/ml). The bone chips were incubated with proteins were washed twice with 1× PBS, All images were captured using an Olympus BX51 microscope system (Olympus).

Statistical Analysis

All data were expressed as mean±standard deviation (SD). OVX Sham and OVX treated groups were compared by Student t-test. In other parts two-way and one-way ANOVA were used. A P-value less than 0.05 were considered statistically significant.

All publications and patents cited in this specification are herein incorporated by reference as if each individual publication or patent were specifically and individually indicated to be incorporated by reference and are incorporated herein by reference to disclose and describe the methods and materials in connection with which the publications are cited.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

It should be understood that although the present invention has been specifically disclosed by certain aspects, embodiments, and optional features, modification, improvement, and variation of such aspects, embodiments, and optional features can be resorted to by those skilled in the art, and that such modifications, improvements, and variations are considered to be within the scope of this disclosure.

What is claimed is:

1-27. (canceled)

28. A method of treating a fracture, the method comprising administering to a subject having a fracture an anabolic agent fusion protein, wherein the anabolic agent fusion protein comprises platelet derived growth factor (PDGF) or fibroblast growth factor (FGF) fused to a Asp Ser Ser tripeptide (DSS) repeat sequence.

29. The method of claim 28, wherein the anabolic agent fusion protein comprises PDGF fused to the DSS repeat sequence with six repeats (DSS6).

30. (canceled)

31. The method of claim 28, wherein the method comprises locally administering the anabolic agent fusion protein to the site of a the fracture.

32. (canceled)

33. The method of claim 31, wherein the anabolic agent fusion protein is administered by a micropump.

34. (canceled)

35. (canceled)

36. The method of claim 28, wherein the anabolic agent fusion protein is administered to accelerates delayed fracture healing.

37. The method of claim 28, wherein a single dose of the anabolic agent fusion protein is administered to the subject.

38. A method of increasing bone growth in a subject, the method comprising administering to the subject a therapeutically effective amount of engineered cells that express an anabolic agent fusion protein, wherein the anabolic agent fusion protein comprises PDGF or FGF fused to a DSS repeat sequence.

39. The method of claim 38, wherein the anabolic agent fusion protein comprises PDGF fused to DSS6.

40. The method of claim 38, wherein the engineered cell is a stem cell.

41. The method of claim 40, wherein the stem cell is a hematopoietic stem cell or a mesenchymal stem cell.

42. The method of claim 38, wherein the engineered cell is autologous to the subject.

43. The method of claim 38, wherein the engineered cell is administered locally at a site of bone loss.

44. (canceled)

45. The method of claim 38, wherein the engineered cell is administered systemically.

46. The method of claim 45, wherein the engineered cell is administered intravenously.

47. The method of claim 38, wherein the subject has osteoporosis.

48. The method of claim 47, wherein the subject has severe osteoporosis.

49. A method of increasing bone growth in a subject, the method comprising locally administering to the subject an anabolic agent fusion protein that comprises PDGF or FGF fused to a DSS repeat sequence, wherein the anabolic agent fusion protein is locally administered to a site of bone loss.

50. The method of claim 49, wherein the anabolic agent fusion protein comprises PDGF fused to DSS6.

51. (canceled)

52. The method of claim 49, wherein the anabolic agent fusion protein is administered by injection.

53. The method of claim 49, wherein the subject has osteoporosis.
